(19) United States
(12) Patent Application Publication
(10) Pub. No.: US 2024/0312642 A1
(43) Pub. Date: Sep. 19, 2024

(54) METHODS AND SYSTEMS FOR OPTIMIZED CUSTOMER RELATIONSHIP MANAGEMENT IN HEALTHCARE

(71) Applicant: Whiterabbit.ai Inc., Santa Clara, CA (US)

(72) Inventors: Rakesh Mathur, Santa Clara, CA (US); Jason Su, Santa Clara, CA (US); Terri Mahannah, Santa Clara, CA (US); Hrishikesh Deshpande, Mumbai (IN); Siddhartha Chattopadhyay, Santa Clara, CA (US); Karan Pahwa, Santa Clara, CA (US); Roxanna Betancourt, Santa Clara, CA (US); Devesh Varshney, Mumbai (IN); Hemlata Malav, Mumbai (IN); Sadanand Singh, Santa Clara, CA (US); Mohd Javed Khan, Mumbai (IN); Saurav Bansal, Mumbai (IN); Saurabh Agarwal, Mumbai (IN); Chiran Doshi, Mumbai (IN); Sanjay Dalsania, Mumbai (IN); Yash Savla, Mumbai (IN); Divya Mamgai, Mumbai (IN); Keshav Raghu, Mumbai (IN); Aasim Ansari, Mumbai (IN)

(21) Appl. No.: 18/378,325

(22) Filed: Oct. 10, 2023

Related U.S. Application Data
(63) Continuation of application No. PCT/US2022/024224, filed on Apr. 11, 2022.

(30) Foreign Application Priority Data
Apr. 12, 2021 (IN) 202121016977

(51) Int. Cl. G16H 80/00; G06T 7/00; G16H 10/60; G16H 40/20; G16H 50/20

(52) U.S. Cl. CPC G16H 80/00; G06T 7/0012; G16H 10/60; G16H 40/20; G16H 50/20; G06T 2207/30004

(57) ABSTRACT

The present disclosure provides methods, systems, and media for optimized customer relationship management. A method for identifying subjects for a clinical procedure may comprise (a) retrieving, from a computer database, a first set of subject records, wherein the first set of subject records corresponds to a first set of subjects that are candidates for the clinical procedure; (b) processing the first set of subject records using a trained machine learning algorithm to generate a second set of subject records; and (c) electronically outputting the second set of subject records.

### CROSS REFERENCE

This application is a continuation of International Application No. PCT/US2022/024224, filed Apr. 11, 2022, which claims the benefit of India (IN) Patent Application 202121016977, filed Apr. 12, 2021, each of which is incorporated by reference herein in its entirety.

### BACKGROUND

The clinical use of medical imaging examinations, such as routine screening or follow-up care for cancer, has demonstrated significant benefits in reducing mortality, improving prognoses, and lowering treatment costs. Despite these demonstrated benefits, adoption rates for screening or follow-up care (e.g., mammography) are hindered, in part, by ineffective or inefficient outreach processes.

### SUMMARY

The present disclosure provides methods, systems, and media for optimized customer relationship management in healthcare. For example, subjects may include subjects with a disease, disorder, or abnormal condition (e.g., cancer) and subjects without a disease, disorder, or abnormal condition (e.g., asymptomatic subjects undergoing routine screening exams). Subjects may be candidates for receiving clinical procedures, such as a screening exam. The screening exam may be for a cancer such as, for example, breast cancer.

In an aspect, the present disclosure provides a computer-implemented method for identifying subjects for a clinical procedure, comprising: (a) retrieving, from a computer database, a first set of subject records, wherein the first set of subject records corresponds to a first set of subjects that are candidates for the clinical procedure; (b) processing the first set of subject records using a trained machine learning algorithm to generate a second set of subject records, wherein the second set of subject records is a subset of the first set of subject records, wherein the second set of subject records corresponds to a second set of subjects, which second set of subjects is a subset of the first set of subjects; and (c) electronically outputting the second set of subject records.

In some embodiments, the clinical procedure is a diagnostic test, a prognostic test, a therapeutic intervention, or a prophylactic intervention. In some embodiments, the clinical procedure is a diagnostic test for a clinical disease, disorder, or condition. In some embodiments, the clinical disease, disorder, or condition is cancer. In some embodiments, the cancer is breast cancer.

In some embodiments, the diagnostic test comprises obtaining a medical image of a test subject, and analyzing the medical image sample to determine a diagnosis of the clinical disease, disorder, or condition. In some embodiments, the diagnostic test comprises obtaining a biological sample from a test subject, and assaying the biological sample to determine a diagnosis of the clinical disease, disorder, or condition.

In some embodiments, the first set of subjects previously received the clinical procedure. In some embodiments, the second set of subjects is prioritized over other subjects of the first set of subjects with respect to the clinical procedure.

In some embodiments, (b) comprises processing a set of features of the first set of subject records using the trained machine learning algorithm to determine a score for each of at least a subset of the first set of subject records, and generating the second set of subject records based at least in part on the scores for the at least the subset of the first set of subject records.

In some embodiments, the score for a given subject record is indicative of a likelihood of compliance of a given subject with receiving the clinical procedure.

In some embodiments, a given subject record is selected for inclusion in the second set of subject records when the given subject record (i) has a score that is greater than a first pre-determined threshold and/or (ii) has a score that is less than a second pre-determined threshold.

In some embodiments, the set of features is selected from the group consisting of demographic characteristics, clinical characteristics, clinical history, and history of past outreach. In some embodiments, the demographic characteristics are selected from the group consisting of age, gender, race, ethnicity, occupation, income, and education level. In some embodiments, the demographic characteristics, clinical characteristics, clinical history, and/or history of past outreach are obtained from electronic medical records of subjects. In some embodiments, the demographic characteristics, clinical characteristics, clinical history, and/or history of past outreach obtained from electronic medical records of subjects are joined or enriched with external data sources (e.g., publicly available data).

In some embodiments, the trained machine learning algorithm comprises a supervised machine learning algorithm. In some embodiments, the supervised machine learning algorithm comprises a deep learning algorithm, a support vector machine (SVM), a neural network, or a Random Forest.

In some embodiments, the method further comprises recruiting subjects to receive the clinical procedure based at least in part on the second set of subject records. In some embodiments, recruiting the subjects comprises assigning the subjects to receive the clinical procedure at a clinical site. In some embodiments, the method further comprises selecting the clinical site from among a plurality of clinical sites based at least in part on an availability or load of the clinical site. In some embodiments, recruiting the subjects comprises assigning the subjects to receive one of a set of alternative clinical procedures and/or treatments (e.g., selected from the set based on external data and/or bidding of the providers and/or vendors of the procedures and/or treatments). In some embodiments, recruiting the subjects comprises determining an optimal frequency, cadence, communication channel, and/or content for performing outreach attempts to the subjects. In some embodiments, recruiting the subjects comprises determining an optimal frequency, cadence, communication channel, and/or content for temporally shifting patient populations. In some embodiments, the method further comprises transmitting an alert, notification, phone call, or reminder to at least one subject of the second set of subjects to have the clinical procedure performed.

In another aspect, the present disclosure provides a computer system for prioritizing subjects for a clinical procedure, comprising: a database that is configured to store a first set of subject records, wherein the first set of subject records corresponds to a first set of subjects that are candidates for the clinical procedure; and one or more computer processors operatively coupled to the database, wherein the one or more computer processors are individually or collectively programmed to: (i) process the first set of subject records using a trained machine learning algorithm to generate a second set of subject records, wherein the second set of subject records is a subset of the first set of subject records, wherein the second set of subject records corresponds to a second set of subjects, which second set of subjects is a subset of the first set of subjects; and (ii) electronically output the second set of subject records.

In some embodiments, the clinical procedure is a diagnostic test, a prognostic test, a therapeutic intervention, or a prophylactic intervention. In some embodiments, the clinical procedure is a diagnostic test for a clinical disease, disorder, or condition. In some embodiments, the clinical disease, disorder, or condition is cancer. In some embodiments, the cancer is breast cancer.

In some embodiments, the diagnostic test comprises obtaining a medical image of a test subject, and analyzing the medical image sample to determine a diagnosis of the clinical disease, disorder, or condition. In some embodiments, the diagnostic test comprises obtaining a biological sample from a test subject, and assaying the biological sample to determine a diagnosis of the clinical disease, disorder, or condition.

In some embodiments, the first set of subjects previously received the clinical procedure. In some embodiments, the second set of subjects is prioritized over other subjects of the first set of subjects with respect to the clinical procedure.

In some embodiments, (i) comprises processing a set of features of the first set of subject records using the trained machine learning algorithm to determine a score for each of at least a subset of the first set of subject records, and generating the second set of subject records based at least in part on the scores for the at least the subset of the first set of subject records.

In some embodiments, the score for a given subject record is indicative of a likelihood of compliance of a given subject with receiving the clinical procedure.

In some embodiments, a given subject record is selected for inclusion in the second set of subject records when the given subject record (i) has a score that is greater than a first pre-determined threshold and/or (ii) has a score that is less than a second pre-determined threshold.

In some embodiments, the set of features is selected from the group consisting of demographic characteristics, clinical characteristics, clinical history, and history of past outreach. In some embodiments, the demographic characteristics are selected from the group consisting of age, gender, race, ethnicity, occupation, income, and education level. In some embodiments, the demographic characteristics, clinical characteristics, clinical history, and/or history of past outreach are obtained from electronic medical records of subjects. In some embodiments, the demographic characteristics, clinical characteristics, clinical history, and/or history of past outreach obtained from electronic medical records of subjects are joined or enriched with external data sources (e.g., publicly available data).

In some embodiments, the trained machine learning algorithm comprises a supervised machine learning algorithm. In some embodiments, the supervised machine learning algorithm comprises a deep learning algorithm, a support vector machine (SVM), a neural network, or a Random Forest.

In some embodiments, the one or more computer processors are individually or collectively programmed to further recruit subjects to receive the clinical procedure based at least in part on the second set of subject records. In some embodiments, recruiting the subjects comprises assigning the subjects to receive the clinical procedure at a clinical site. In some embodiments, the one or more computer processors are individually or collectively programmed to further select the clinical site from among a plurality of clinical sites based at least in part on an availability or load of the clinical site. In some embodiments, recruiting the subjects comprises assigning the subjects to receive one of a set of alternative clinical procedures and/or treatments (e.g., selected from the set based on external data and/or bidding of the providers and/or vendors of the procedures and/or treatments). In some embodiments, recruiting the subjects comprises determining an optimal frequency, cadence, communication channel, and/or content for performing outreach attempts to the subjects. In some embodiments, recruiting the subjects comprises determining an optimal frequency, cadence, communication channel, and/or content for temporally shifting patient populations. In some embodiments, the one or more computer processors are individually or collectively programmed to further transmit an alert, notification, phone call, or reminder to at least one subject of the second set of subjects to have the clinical procedure performed.

In another aspect, the present disclosure provides a non-transitory computer readable medium comprising machine-executable code that, upon execution by one or more computer processors, implements a method for identifying subjects for a clinical procedure, the method comprising: (a) retrieving, from a computer database, a first set of subject records, wherein the first set of subject records corresponds to a first set of subjects that are candidates for the clinical procedure; (b) processing the first set of subject records using a trained machine learning algorithm to generate a second set of subject records, wherein the second set of subject records is a subset of the first set of subject records, wherein the second set of subject records corresponds to a second set of subjects, which second set of subjects is a subset of the first set of subjects; and (c) electronically outputting the second set of subject records.

In some embodiments, the clinical procedure is a diagnostic test, a prognostic test, a therapeutic intervention, or a prophylactic intervention. In some embodiments, the clinical procedure is a diagnostic test for a clinical disease, disorder, or condition. In some embodiments, the clinical disease, disorder, or condition is cancer. In some embodiments, the cancer is breast cancer.

In some embodiments, the diagnostic test comprises obtaining a medical image of a test subject, and analyzing the medical image sample to determine a diagnosis of the clinical disease, disorder, or condition. In some embodiments, the diagnostic test comprises obtaining a biological sample from a test subject, and assaying the biological sample to determine a diagnosis of the clinical disease, disorder, or condition.

In some embodiments, the first set of subjects previously received the clinical procedure. In some embodiments, the second set of subjects is prioritized over other subjects of the first set of subjects with respect to the clinical procedure.

In some embodiments, (b) comprises processing a set of features of the first set of subject records using the trained machine learning algorithm to determine a score for each of at least a subset of the first set of subject records, and generating the second set of subject records based at least in part on the scores for the at least the subset of the first set of subject records.

In some embodiments, the score for a given subject record is indicative of a likelihood of compliance of a given subject with receiving the clinical procedure.

In some embodiments, a given subject record is selected for inclusion in the second set of subject records when the given subject record (i) has a score that is greater than a first pre-determined threshold and/or (ii) has a score that is less than a second pre-determined threshold.

In some embodiments, the set of features is selected from the group consisting of demographic characteristics, clinical characteristics, clinical history, and history of past outreach. In some embodiments, the demographic characteristics are selected from the group consisting of age, gender, race, ethnicity, occupation, income, and education level. In some embodiments, the demographic characteristics, clinical characteristics, clinical history, and/or history of past outreach are obtained from electronic medical records of subjects. In some embodiments, the demographic characteristics, clinical characteristics, clinical history, and/or history of past outreach obtained from electronic medical records of subjects are joined or enriched with external data sources (e.g., publicly available data).

In some embodiments, the trained machine learning algorithm comprises a supervised machine learning algorithm. In some embodiments, the supervised machine learning algorithm comprises a deep learning algorithm, a support vector machine (SVM), a neural network, or a Random Forest.

In some embodiments, the method further comprises recruiting subjects to receive the clinical procedure based at least in part on the second set of subject records. In some embodiments, recruiting the subjects comprises assigning the subjects to receive the clinical procedure at a clinical site. In some embodiments, the method further comprises selecting the clinical site from among a plurality of clinical sites based at least in part on an availability or load of the clinical site. In some embodiments, recruiting the subjects comprises assigning the subjects to receive one of a set of alternative clinical procedures and/or treatments (e.g., selected from the set based on external data and/or bidding of the providers and/or vendors of the procedures and/or treatments). In some embodiments, recruiting the subjects comprises determining an optimal frequency, cadence, communication channel, and/or content for performing outreach attempts to the subjects. In some embodiments, recruiting the subjects comprises determining an optimal frequency, cadence, communication channel, and/or content for temporally shifting patient populations. In some embodiments, the method further comprises transmitting an alert, notification, phone call, or reminder to at least one subject of the second set of subjects to have the clinical procedure performed.

In another aspect, the present disclosure provides a computer-implemented method for identifying subjects for a clinical procedure, comprising: (a) retrieving, from a computer database, a first set of subject records, wherein the first set of subject records corresponds to a first set of subjects that are candidates for the clinical procedure; (b) processing the first set of subject records using a trained machine learning algorithm to identify a second set of subjects and a second set of subject records associated with the second set of subjects, wherein the second set of subjects is a ranked subset of the first set of subjects, and wherein the second set of subject records is a subset of the first set of subject records; and (c) generating an electronic output that identifies one or more subjects from the second set of subjects to receive the clinical procedure.

In some embodiments, the clinical procedure is a target screening exam, a diagnostic test, prognostic test, a therapeutic intervention, or a prophylactic intervention. In some embodiments, the clinical procedure is a diagnostic test for a clinical disease, disorder, or condition. In some embodiments, the clinical disease, disorder, or condition is cancer. In some embodiments, the cancer is breast cancer. In some embodiments, the diagnostic test comprises obtaining a medical image of a test subject, and analyzing the medical image to determine a diagnosis of the clinical disease, disorder, or condition. In some embodiments, the diagnostic test comprises obtaining a biological sample from a test subject, and assaying the biological sample to determine a diagnosis of the clinical disease, disorder, or condition.

In some embodiments, the first set of subjects previously received the clinical procedure. In some embodiments, the second set of subjects is prioritized over other subjects of the first set of subjects with respect to the clinical procedure.

In some embodiments, (b) comprises processing a set of features of the first set of subject records using the trained machine learning algorithm to determine a score for each of at least a subset of the first set of subject records, and generating the second set of subject records based at least in part on the scores for the at least the subset of the first set of subject records. In some embodiments, the score for a given subject record is indicative of a likelihood of compliance of a given subject with receiving the clinical procedure. In some embodiments, a given subject record is selected for inclusion in the second set of subject records when the given subject record (i) has a score that is greater than a first pre-determined threshold and/or (ii) has a score that is less than a second pre-determined threshold. In some embodiments, the set of features is selected from the group consisting of demographic characteristics, clinical characteristics, clinical history, and history of past outreach. In some embodiments, the demographic characteristics are selected from the group consisting of age, gender, race, ethnicity, occupation, income, and education level. In some embodiments, the demographic characteristics, clinical characteristics, clinical history, and/or history of past outreach are obtained from electronic medical records of subjects.

In some embodiments, the trained machine learning algorithm comprises a supervised machine learning algorithm. In some embodiments, the supervised machine learning algorithm comprises a deep learning algorithm, a support vector machine (SVM), a neural network, or a Random Forest.

In some embodiments, the method may further comprise recruiting subjects to receive the clinical procedure based at least in part on the second set of subject records. In some embodiments, recruiting the subjects comprises assigning the subjects to receive the clinical procedure at a clinical site. In some embodiments, the method may further comprise selecting the clinical site from among a plurality of clinical sites based at least in part on an availability or load of the clinical site. In some embodiments, recruiting the subjects comprises assigning the subjects to receive one of a set of alternative clinical procedures and/or treatments. In some embodiments, recruiting the subjects comprises determining an optimal frequency, cadence, communication channel, and/or content for performing outreach attempts to the subjects. In some embodiments, recruiting the subjects comprises determining an optimal frequency, cadence, communication channel, and/or content for temporally shifting patient populations.

In some embodiments, the method may further comprise transmitting an alert, notification, phone call, or reminder to at least one subject of the second set of subjects to have the clinical procedure performed. The clinical procedure may comprise any procedure, operation, or health-care related event as described herein.

In another aspect, the present disclosure provides a computer system for identifying subjects for a clinical procedure, comprising: a database that is configured to store a first set of subject records, wherein the first set of subject records corresponds to a first set of subjects that are candidates for the clinical procedure; and one or more computer processors operatively coupled to the database, wherein the one or more computer processors are individually or collectively programmed to: (i) process the first set of subject records using a trained machine learning algorithm to identify a second set of subjects and a second set of subject records associated with the second set of subjects, wherein the second set of subjects is a ranked subset of the first set of subjects, and wherein the second set of subject records is a subset of the first set of subject records; and (ii) generate an electronic output that identifies one or more subjects from the second set of subjects to receive the clinical procedure.

In some embodiments, the clinical procedure is a target screening exam, a diagnostic test, a prognostic test, a therapeutic intervention, or a prophylactic intervention. In some embodiments, the clinical procedure is a diagnostic test for a clinical disease, disorder, or condition. In some embodiments, the clinical disease, disorder, or condition is cancer. In some embodiments, the cancer is breast cancer. In some embodiments, the diagnostic test comprises obtaining a medical image of a test subject, and analyzing the medical image sample to determine a diagnosis of the clinical disease, disorder, or condition. In some embodiments, the diagnostic test comprises obtaining a biological sample from a test subject, and assaying the biological sample to determine a diagnosis of the clinical disease, disorder, or condition.

In some embodiments, the first set of subjects previously received the clinical procedure. In some embodiments, the second set of subjects is prioritized over other subjects of the first set of subjects with respect to the clinical procedure.

In some embodiments, the one or more processors are configured to process a set of features of the first set of subject records using the trained machine learning algorithm to determine a score for each of at least a subset of the first set of subject records, and generate the second set of subject records based at least in part on the scores for the at least the subset of the first set of subject records. In some embodiments, the score for a given subject record is indicative of a likelihood of compliance of a given subject with receiving the clinical procedure. In some embodiments, a given subject record is selected for inclusion in the second set of subject records when the given subject record (i) has a score that is greater than a first pre-determined threshold and/or (ii) has a score that is less than a second pre-determined threshold. In some embodiments, the set of features is selected from the group consisting of demographic characteristics, clinical characteristics, clinical history, and history of past outreach. In some embodiments, the demographic characteristics are selected from the group consisting of age, gender, race, ethnicity, occupation, income, and education level. In some embodiments, the clinical characteristics and/or the clinical history are obtained from electronic medical record of subjects.

In some embodiments, the trained machine learning algorithm comprises a supervised machine learning algorithm. In some embodiments, the supervised machine learning algorithm comprises a deep learning algorithm, a support vector machine (SVM), a neural network, or a Random Forest.

In some embodiments, the one or more computer processors are individually or collectively programmed to further recruit subjects to receive the clinical procedure based at least in part on the second set of subject records. In some embodiments, recruiting the subjects comprises assigning the subjects to receive the clinical procedure at a clinical site. In some embodiments, the one or more computer processors are individually or collectively programmed to further select the clinical site from among a plurality of clinical sites based at least in part on an availability or load of the clinical site. In some embodiments, recruiting the subjects comprises assigning the subjects to receive one of a set of alternative clinical procedures and/or treatments. In some embodiments, recruiting the subjects comprises determining an optimal frequency, cadence, communication channel, and/or content for performing outreach attempts to the subjects. In some embodiments, recruiting the subjects comprises determining an optimal frequency, cadence, communication channel, and/or content for temporally shifting patient populations.

In some embodiments, the one or more computer processors are individually or collectively programmed to further transmit an alert, notification, or reminder to at least one subject of the second set of subjects to have the clinical procedure performed. The clinical procedure may comprise any procedure, operation, or health-care related event as described herein.

In another aspect, the present disclosure provides a non-transitory computer readable medium comprising machine-executable code that, upon execution by one or more computer processors, implements a method for identifying subjects for a clinical procedure. The method may comprise (a) retrieving, from a computer database, a first set of subject records, wherein the first set of subject records corresponds to a first set of subjects that are candidates for the clinical procedure; (b) processing the first set of subject records using a trained machine learning algorithm to identify a second set of subjects and a second set of subject records associated with the second set of subjects, wherein the second set of subjects is a ranked subset of the first set of subjects, and wherein the second set of subject records is a subset of the first set of subject records; and (c) generating an electronic output that identifies one or more subjects from the second set of subjects to receive the clinical procedure.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates an example workflow of a method for prioritizing subjects for a clinical procedure.

FIG. 2 illustrates a computer system that is programmed or otherwise configured to implement methods provided herein.

FIG. 3 shows an example of a spreadsheet-based outreach performed using the ACT CRM system. Worklists are pulled from an existing RIS system. Caller expectations may be set at a pre-determined level (e.g., 60 calls per day). This outreach may be a manual process.

FIG. 4 shows an example of an automated outreach performed using the ACT CRM system. Automated worklists may be generated by the ACT system to implement a nine-stage outreach program (e.g., 3 calls, and the rest texts).

FIG. 5 shows an example of an automated outreach performed using the ACT CRM system.

FIG. 6A shows an example of the integration of various aspects into the ACT system, including an outreach program, call center metrics, engineering, and AI/ML models.

FIG. 6B shows an example of an ACT model pipeline. With ACT, callers contact patients to come in for a periodic exam. The model triages patients for callers to focus outreach on a subset of patients that they can most affect.

FIG. 6C shows an example of a probability of compliance of patients based on their days since last screening visit.

FIG. 6D shows an example of a probability of compliance of patients based on their recency.

FIG. 6E shows an example of a probability of compliance of patients based on their weighted compliance rate.

FIGS. 6F-6H show an ROC curve (FIG. 6F) and confusion matrices (FIGS. 6G-6H) for prediction of patient self-compliance based on weighted compliance rate.

FIGS. 6I-6K show an ROC curve (FIG. 6I) and confusion matrices (FIGS. 6J-6K) for prediction of whether a patient is likely to convert via the ACT CRM outreach.

FIGS. 7A-7S show example screenshots of a user interface of a CRM system. Patients are prioritized based on the predictions of AI/ML models.

FIGS. 8A-8B shows an example of the ACT CRM system applied to mammography.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular form “a”, “an”, and “the” include plural references unless the context clearly dictates otherwise. For example, the term “a nucleic acid” includes a plurality of nucleic acids, including mixtures thereof.

As used herein, the term “subject,” generally refers to an entity or a medium that has testable or detectable genetic information. A subject can be a person, individual, or patient. A subject can be a vertebrate, such as, for example, a mammal. Non-limiting examples of mammals include humans, simians, farm animals, sport animals, rodents, and pets. The subject can be a person that has a disease, disorder, or abnormal condition (e.g., cancer) or is suspected of having a disease, disorder, or abnormal condition. The subject may be displaying a symptom(s) indicative of a health or physiological state or condition of the subject, such as a cancer (e.g., breast cancer) of the subject. As an alternative, the subject can be asymptomatic with respect to such health or physiological state or condition.

The clinical use of medical imaging examinations, such as routine screening or follow-up care for cancer, has demonstrated significant benefits in reducing mortality, improving prognoses, and lowering treatment costs. Despite these demonstrated benefits, adoption rates for screening or follow-up care (e.g., mammography) are hindered, in part, by ineffective or inefficient outreach processes.

The present disclosure provides methods, systems, and media for optimized customer relationship management. For example, subjects may include subjects with a disease, disorder, or abnormal condition (e.g., cancer) and subjects without a disease, disorder, or abnormal condition (e.g., asymptomatic subjects undergoing routine screening exams). Subjects may be candidates for receiving clinical procedures, such as screening. The screening may be for a cancer such as, for example, breast cancer.

In an aspect, the present disclosure provides a computer-implemented method for identifying subjects for a clinical procedure, comprising: (a) retrieving, from a computer database, a first set of subject records, wherein the first set of subject records corresponds to a first set of subjects that are candidates for the clinical procedure; (b) processing the first set of subject records using a trained machine learning algorithm to identify a second set of subjects and a second set of subject records associated with the second set of subjects, wherein the second set of subjects is a ranked subset of the first set of subjects, and wherein the second set of subject records is a subset of the first set of subject records; and (c) electronically outputting the second set of subject records.

In some embodiments, the clinical procedure is a target screening exam, a diagnostic test, a prognostic test, a therapeutic intervention, or a prophylactic intervention. In some embodiments, the clinical procedure is a diagnostic test for a clinical disease, disorder, or condition. In some embodiments, the clinical disease, disorder, or condition is cancer. In some embodiments, the cancer is breast cancer.

In some embodiments, the diagnostic test comprises obtaining a medical image of a test subject, and analyzing the medical image sample to determine a diagnosis of the clinical disease, disorder, or condition. In some embodiments, the diagnostic test comprises obtaining a biological sample from a test subject, and assaying the biological sample to determine a diagnosis of the clinical disease, disorder, or condition.

In some embodiments, the first set of subjects previously received the clinical procedure. In some embodiments, the second set of subjects is prioritized over other subjects of the first set of subjects with respect to the clinical procedure.

In some embodiments, (b) comprises processing a set of features of the first set of subject records using the trained machine learning algorithm to determine a score for each of at least a subset of the first set of subject records, and generating the second set of subject records based at least in part on the scores for the at least the subset of the first set of subject records.

The second set of subjects may be a ranked subset of the first set of subjects. In some cases, the subjects in the second set of subjects may be the same as the subjects in the first set of subjects. In such cases, the subjects in the second set of subjects may comprise a listing, an arrangement, or a grouping of the first set of subjects according to a rank or a score. In some cases, the subjects in the second set of subjects may comprise a subset of the subjects in the first set of subjects. In such cases, the subjects in the second set of subjects may comprise a listing, an arrangement, or a grouping of the subset of the subjects in the first set of subjects according to a rank or a score. In some embodiments, the rank or score may be associated with a likelihood or a probability of an event occurring (e.g., the likelihood of a patient answering or responding to a communication at a certain time of day, or likelihood of compliance with a treatment).

In some embodiments, the rank or score for a given subject or subject record is indicative of a likelihood of compliance of a given subject with receiving the clinical procedure.

In some embodiments, a given subject record is selected for inclusion in the second set of subject records when the given subject record (i) has a score that is greater than a first pre-determined threshold and/or (ii) has a score that is less than a second pre-determined threshold.

In some embodiments, the set of features is selected from the group consisting of demographic characteristics, clinical characteristics, clinical history, and history of past outreach. In some embodiments, the demographic characteristics are selected from the group consisting of age, gender, race, ethnicity, occupation, income, and education level. In some embodiments, the clinical characteristics and/or the clinical history are obtained from electronic medical record of subjects.

In some embodiments, the trained machine learning algorithm comprises a supervised machine learning algorithm. In some embodiments, the supervised machine learning algorithm comprises a deep learning algorithm, a support vector machine (SVM), a neural network, or a Random Forest.

In some embodiments, the method further comprises recruiting subjects to receive the clinical procedure based at least in part on the second set of subject records. In some embodiments, recruiting the subjects comprises assigning the subjects to receive the clinical procedure at a clinical site. In some embodiments, the method further comprises selecting the clinical site from among a plurality of clinical sites based at least in part on an availability or load of the clinical site. In some embodiments, the method further comprises transmitting an alert, notification, or reminder to at least one subject of the second set of subjects to have the clinical procedure performed.

In another aspect, the present disclosure provides a computer system for prioritizing subjects for a clinical procedure, comprising: a database that is configured to store a first set of subject records, wherein the first set of subject records corresponds to a first set of subjects that are candidates for the clinical procedure; and one or more computer processors operatively coupled to the database, wherein the one or more computer processors are individually or collectively programmed to: (i) process the first set of subject records using a trained machine learning algorithm to generate a second set of subject records, wherein the second set of subject records is a subset of the first set of subject records, wherein the second set of subject records corresponds to a second set of subjects, which second set of subjects is a subset of the first set of subjects; and (ii) electronically output the second set of subject records.

In some embodiments, the clinical procedure is a diagnostic test, a prognostic test, a therapeutic intervention, or a prophylactic intervention. In some embodiments, the clinical procedure is a diagnostic test for a clinical disease, disorder, or condition. In some embodiments, the clinical disease, disorder, or condition is cancer. In some embodiments, the cancer is breast cancer.

In some embodiments, the diagnostic test comprises obtaining a medical image of a test subject, and analyzing the medical image sample to determine a diagnosis of the clinical disease, disorder, or condition. In some embodiments, the diagnostic test comprises obtaining a biological sample from a test subject, and assaying the biological sample to determine a diagnosis of the clinical disease, disorder, or condition.

In some embodiments, the first set of subjects previously received the clinical procedure. In some embodiments, the second set of subjects is prioritized over other subjects of the first set of subjects with respect to the clinical procedure.

In some embodiments, (i) comprises processing a set of features of the first set of subject records using the trained machine learning algorithm to determine a score for each of at least a subset of the first set of subject records, and generating the second set of subject records based at least in part on the scores for the at least the subset of the first set of subject records.

In some embodiments, the score for a given subject record is indicative of a likelihood of compliance of a given subject with receiving the clinical procedure.

In some embodiments, a given subject record is selected for inclusion in the second set of subject records when the given subject record (i) has a score that is greater than a first pre-determined threshold and/or (ii) has a score that is less than a second pre-determined threshold.

In some embodiments, the set of features is selected from the group consisting of demographic characteristics, clinical characteristics, clinical history, and history of past outreach. In some embodiments, the demographic characteristics are selected from the group consisting of age, gender, race, ethnicity, occupation, income, and education level. In some embodiments, the clinical characteristics and/or the clinical history are obtained from electronic medical record of subjects.

In some embodiments, the trained machine learning algorithm comprises a supervised machine learning algorithm. In some embodiments, the supervised machine learning algorithm comprises a deep learning algorithm, a support vector machine (SVM), a neural network, or a Random Forest.

In some embodiments, the one or more computer processors are individually or collectively programmed to further recruit subjects to receive the clinical procedure based at least in part on the second set of subject records. In some embodiments, recruiting the subjects comprises assigning the subjects to receive the clinical procedure at a clinical site. In some embodiments, the one or more computer processors are individually or collectively programmed to further select the clinical site from among a plurality of clinical sites based at least in part on an availability or load of the clinical site. In some embodiments, the one or more computer processors are individually or collectively programmed to further transmit an alert, notification, or reminder to at least one subject of the second set of subjects to have the clinical procedure performed.

In another aspect, the present disclosure provides a non-transitory computer readable medium comprising machine-executable code that, upon execution by one or more computer processors, implements a method for identifying subjects for a clinical procedure, the method comprising: (a) retrieving, from a computer database, a first set of subject records, wherein the first set of subject records corresponds to a first set of subjects that are candidates for the clinical procedure; (b) processing the first set of subject records using a trained machine learning algorithm to generate a second set of subject records, wherein the second set of subject records is a subset of the first set of subject records, wherein the second set of subject records corresponds to a second set of subjects, which second set of subjects is a subset of the first set of subjects; and (c) electronically outputting the second set of subject records.

In some embodiments, the clinical procedure is a diagnostic test, a prognostic test, a therapeutic intervention, or a prophylactic intervention. In some embodiments, the clinical procedure is a diagnostic test for a clinical disease, disorder, or condition. In some embodiments, the clinical disease, disorder, or condition is cancer. In some embodiments, the cancer is breast cancer.

In some embodiments, the diagnostic test comprises obtaining a medical image of a test subject, and analyzing the medical image sample to determine a diagnosis of the clinical disease, disorder, or condition. In some embodiments, the diagnostic test comprises obtaining a biological sample from a test subject, and assaying the biological sample to determine a diagnosis of the clinical disease, disorder, or condition.

In some embodiments, the first set of subjects previously received the clinical procedure. In some embodiments, the second set of subjects is prioritized over other subjects of the first set of subjects with respect to the clinical procedure.

In some embodiments, (b) comprises processing a set of features of the first set of subject records using the trained machine learning algorithm to determine a score for each of at least a subset of the first set of subject records, and generating the second set of subject records based at least in part on the scores for the at least the subset of the first set of subject records.

In some embodiments, the score for a given subject record is indicative of a likelihood of compliance of a given subject with receiving the clinical procedure.

In some embodiments, a given subject record is selected for inclusion in the second set of subject records when the given subject record (i) has a score that is greater than a first pre-determined threshold and/or (ii) has a score that is less than a second pre-determined threshold.

In some embodiments, the set of features is selected from the group consisting of demographic characteristics, clinical characteristics, clinical history, and history of past outreach. In some embodiments, the demographic characteristics are selected from the group consisting of age, gender, race, ethnicity, occupation, income, and education level. In some embodiments, the clinical characteristics and/or the clinical history are obtained from electronic medical record of subjects.

In some embodiments, the trained machine learning algorithm comprises a supervised machine learning algorithm. In some embodiments, the supervised machine learning algorithm comprises a deep learning algorithm, a support vector machine (SVM), a neural network, or a Random Forest.

In some embodiments, the method further comprises recruiting subjects to receive the clinical procedure based at least in part on the second set of subject records. In some embodiments, recruiting the subjects comprises assigning the subjects to receive the clinical procedure at a clinical site. In some embodiments, the method further comprises selecting the clinical site from among a plurality of clinical sites based at least in part on an availability or load of the clinical site. In some embodiments, the method further comprises transmitting an alert, notification, or reminder to at least one subject of the second set of subjects to have the clinical procedure performed.

In any of the embodiments described herein, the first set of subjects or subject records and/or the second set of subjects or subject records may be ranked or filtered based on one or more characteristics or features of the first or second set of subjects/subject records. In some cases, the one or more characteristics or features may comprise one or more factors that are determined or predicted using machine learning or artificial intelligence. In some cases, the one or more characteristics or features may comprise one or more factors that are determined based on data associated with the first and/or second set of subjects or subject records. In some cases, the one or more factors may correspond to a predicted likelihood for a patient to comply with a treatment or treatment plan, or to answer or respond to a communication at a certain time of day. In other cases, the one or more factors may correspond to patient data such as an age or an income of the patient, information on a clinical procedure for the patient, or information on a health or medical condition of the patient.

In any of the embodiments described herein, one or more processors may be used to run a targeted campaign or search for a target subset of patients or subjects. The targeted campaign or search may be based on one or more characteristics or features of the patients or subjects of interest. In some cases, the targeted campaign or search may produce a list of patients or subjects having a particular characteristic or feature of interest. Such characteristic or feature of interest may be derived from patient or subjects records, or the processing of such patient or subjects records using machine learning or artificial intelligence. In some cases, the list of patients or subjects may comprise a filtered or ranked subset of patients or subjects as described elsewhere herein.

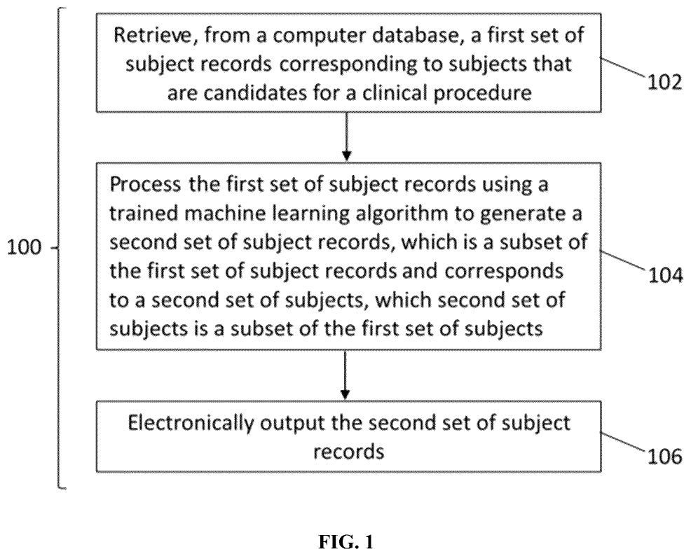

FIG. 1 illustrates an example workflow of a method for radiological data management and visualization, in accordance with disclosed embodiments. In an aspect, the present disclosure provides a method 100 for identifying subjects for a clinical procedure. The method 100 may comprise retrieving, from a computer database, a first set of subject records (as in operation 102). The first set of subject records may correspond to a first set of subjects that are candidates for the clinical procedure. Next, the method 100 may comprise processing the first set of subject records using a trained machine learning algorithm to generate a second set of subject records (as in operation 104). The second set of subject records may be a subset of the first set of subject records. The second set of subject records may correspond to a second set of subjects, which may be a subset of the first set of subjects. Next, the method 100 may comprise electronically outputting the second set of subject records (as in operation 106).

The subject records may be associated with electronic health record or electronic medical record information, such as medical images of subjects. For example, a set of one or more medical images may be obtained or derived from a human subject (e.g., a patient). The medical images may be stored in a database, such as a computer server (e.g., cloud-based server), a local server, a local computer, or a mobile device (such as smartphone or tablet)). The medical images may be obtained from a subject with a disease, disorder, or abnormal condition, from a subject that is suspected of having the disease, disorder, or abnormal condition, or from a subject that does not have or is not suspected of having the disease, disorder, or abnormal condition.

The medical images may be taken before and/or after treatment of a subject with a disease, disorder, or abnormal condition. Medical images may be obtained from a subject during a treatment or a treatment regime. Multiple sets of medical images may be obtained from a subject to monitor the effects of the treatment over time. The medical images may be taken from a subject known or suspected of having a disease, disorder, or abnormal condition (e.g., cancer such as breast cancer) for which a definitive positive or negative diagnosis is not available via clinical tests. The medical images may be taken from a subject suspected of having a disease, disorder, or abnormal condition. The medical images may be taken from a subject experiencing unexplained symptoms, such as fatigue, nausea, weight loss, aches and pains, weakness, or bleeding. The medical images may be taken from a subject having explained symptoms. The medical images may be taken from a subject at risk of developing a disease, disorder, or abnormal condition due to factors such as familial history, age, hypertension or pre-hypertension, diabetes or pre-diabetes, overweight or obesity, environmental exposure, lifestyle risk factors (e.g., smoking, alcohol consumption, or drug use), or presence of other risk factors.

The medical images may be acquired using one or more imaging modalities, such as a mammography, a computed tomography (CT) scan, a magnetic resonance imaging (MRI) scan, an ultrasound scan, a chest X-ray, a positron emission tomography (PET) scan, a PET-CT scan, or any combination thereof. The medical images may be pre-processed using image processing techniques to enhance image characteristics (e.g., contrast, brightness, sharpness), remove noise or artifacts, filter frequency ranges, compress the images to a small file size, or sample or crop the images. The medical images may be deconstructed or reconstructed (e.g., to create a 3-D rendering from a plurality of 2-D images).

### Trained Algorithms

Methods and systems of the present disclosure may use trained algorithms (e.g., trained machine learning models or classifiers). The trained algorithm may be configured to process a set of features of a set of subject records (e.g., corresponding to a set of subjects or patients) to generate a score indicative of a likelihood of conversion of the subject (e.g., successful compliance with receiving a clinical procedure). The trained algorithm may be configured to generate the outputs with an accuracy of at least about 50%, at least about 55%, at least about 60%, at least about 65%, at least about 70%, at least about 75%, at least about 80%, at least about 85%, at least about 90%, at least about 95%, at least about 96%, at least about 97%, at least about 98%, at least about 99%, or more than 99%.

The trained algorithm may comprise a supervised machine learning algorithm. The trained algorithm may comprise a classification and regression tree (CART) algorithm. The supervised machine learning algorithm may comprise, for example, a Random Forest, a support vector machine (SVM), a neural network (e.g., a deep neural network (DNN)), or a deep learning algorithm. The trained algorithm may comprise an unsupervised machine learning algorithm.

The trained algorithm may be configured to accept a plurality of input variables and to produce one or more output values based on the plurality of input variables. The plurality of input variables may comprise features extracted from one or more datasets comprising subject records of a set of subjects. For example, an input variable may comprise one or more characteristics of a set of subjects, such as: demographic characteristics, clinical characteristics, clinical history, and history of past outreach. In some embodiments, the demographic characteristics are selected from the group consisting of age, gender, race, ethnicity, occupation, income, and education level. In some embodiments, the clinical characteristics and/or the clinical history are obtained from electronic medical record of subjects.

The plurality of input variables may also include clinical health data of a subject. In some embodiments, the clinical health data comprises one or more quantitative measures of the subject, such as age, weight, height, body mass index (BMI), blood pressure, heart rate, glucose levels. As another example, the clinical health data can comprise one or more categorical measures, such as race, ethnicity, history of medication or other clinical treatment, history of tobacco use, history of alcohol consumption, daily activity or fitness level, genetic test results, blood test results, imaging results, and screening results.

The trained algorithm may comprise a classifier, such that each of the one or more output values comprises one of a fixed number of possible values (e.g., a linear classifier, a logistic regression classifier, etc.) indicating a classification of the datasets. The trained algorithm may comprise a binary classifier, such that each of the one or more output values comprises one of two values (e.g., {0, 1}, {positive, negative}, {likely, unlikely}, or {high priority, low priority}) indicating a classification of the set of subject records. The trained algorithm may be another type of classifier, such that each of the one or more output values comprises one of more than two values (e.g., {0, 1, 2}, {positive, negative, or indeterminate}, {likely, unlikely, or indeterminate}, or {high priority, low priority, or medium priority}) indicating a classification of the set of subject records. The output values may comprise descriptive labels, numerical values, or a combination thereof. Some of the output values may comprise descriptive labels. Such descriptive labels may provide an identification, indication, or likelihood of successful outreach of the subject. Such descriptive labels may provide an identification of a follow-up clinical procedure, such as diagnostic procedure or treatment for the subject, and may comprise, for example, a therapeutic intervention, a duration of the therapeutic intervention, and/or a dosage of the therapeutic intervention suitable to treat a disease, disorder, or abnormal condition or other condition. Such descriptive labels may provide an identification of secondary clinical tests that may be appropriate to perform on the subject, and may comprise, for example, an imaging test, a blood test, a mammography, a computed tomography (CT) scan, a magnetic resonance imaging (MRI) scan, an ultrasound scan, a chest X-ray, a positron emission tomography (PET) scan, a PET-CT scan, or any combination thereof. As another example, such descriptive labels may provide a prognosis of the disease, disorder, or abnormal condition of the subject. As another example, such descriptive labels may provide a relative assessment of the disease, disorder, or abnormal condition (e.g., an estimated cancer stage or tumor burden) of the subject. Some descriptive labels may be mapped to numerical values, for example, by mapping “positive” to 1 and “negative” to 0.

Some of the output values may comprise numerical values, such as binary, integer, or continuous values. Such binary output values may comprise, for example, {0, 1}, {positive, negative}, {likely, unlikely}, or {high priority, low priority}. Such integer output values may comprise, for example, {0, 1, 2}. Such continuous output values may comprise, for example, a probability value of at least 0 and no more than 1. Such continuous output values may comprise, for example, an un-normalized probability value of at least 0. Such continuous output values may indicate a score indicative of a likelihood of conversion of the subject (e.g., successful compliance with receiving a clinical procedure). Some numerical values may be mapped to descriptive labels, for example, by mapping 1 to “likely” and 0 to “unlikely.”

Some of the output values may be assigned based on one or more cutoff values. For example, a binary classification of subject records may assign an output value of “positive”, “likely”, or 1 if the analysis of the subject record indicates that the subject has at least a 50% likelihood of conversion of the subject (e.g., successful compliance with receiving a clinical procedure). For example, a binary classification of subject records may assign an output value of “negative” or 0 if the analysis of the subject record indicates that the subject has less than a 50% likelihood of conversion of the subject (e.g., successful compliance with receiving a clinical procedure). In this case, a single cutoff value of 50% is used to classify subject record into one of the two possible binary output values. Examples of single cutoff values may include about 1%, about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, and about 99%.

As another example, a classification of subject records may assign an output value of “positive”, “likely”, or 1 if the analysis of the subject record indicates that the subject has a likelihood of conversion of the subject (e.g., successful compliance with receiving a clinical procedure) of at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or more. The classification of subject records may assign an output value of “positive” or 1 if the analysis of the subject record indicates that the subject has a likelihood of conversion of the subject (e.g., successful compliance with receiving a clinical procedure) of more than about 50%, more than about 55%, more than about 60%, more than about 65%, more than about 70%, more than about 75%, more than about 80%, more than about 85%, more than about 90%, more than about 91%, more than about 92%, more than about 93%, more than about 94%, more than about 95%, more than about 96%, more than about 97%, more than about 98%, or more than about 99%.

The classification of subject records may assign an output value of “negative”, “unlikely”, or 0 if the analysis of the subject record indicates that the subject has a likelihood of conversion of the subject (e.g., successful compliance with receiving a clinical procedure) of no more than about 50%, no more than about 45%, no more than about 40%, no more than about 35%, no more than about 30%, no more than about 25%, no more than about 20%, no more than about 15%, no more than about 10%, no more than about 9%, no more than about 8%, no more than about 7%, no more than about 6%, no more than about 5%, no more than about 4%, no more than about 3%, no more than about 2%, or no more than about 1%. The classification of subject records may assign an output value of “negative”, “unlikely”, or 0 if the analysis of the subject record indicates that the subject has a likelihood of conversion of the subject (e.g., successful compliance with receiving a clinical procedure) of less than about 50%, less than about 45%, less than about 40%, less than about 35%, less than about 30%, less than about 25%, less than about 20%, less than about 15%, less than about 10%, less than about 9%, less than about 8%, less than about 7%, less than about 6%, less than about 5%, less than about 4%, less than about 3%, less than about 2%, or less than about 1%.

The classification of subject records may assign an output value of “indeterminate” or 2 if the subject record is not classified as “positive”, “negative”, “likely”, “unlikely”, 1, or 0. In this case, a set of two cutoff values is used to classify subject records into one of the three possible output values. Examples of sets of cutoff values may include {1%, 99%}, {2%, 98%}, {5%, 95%}, {10%, 90%}, {15%, 85%}, {20%, 80%}, {25%, 75%}, {30%, 70%}, {35%, 65%}, {40%, 60%}, and {45%, 55%}. Similarly, sets of n cutoff values may be used to classify subject records into one of n+1 possible output values, where n is any positive integer.

The trained algorithm may be trained with a plurality of independent training samples. Each of the independent training samples may comprise a set of subject records, associated datasets corresponding to the subjects (e.g., health data), and one or more known output values corresponding to the set of subject records (e.g., prior history of compliance). Independent training samples may comprise subject records, and associated datasets and outputs obtained or derived from a plurality of different subjects. Independent training samples may comprise subject records and associated datasets and outputs obtained at a plurality of different time points from the same subject (e.g., on a regular basis such as weekly, biweekly, or monthly). Independent training samples may be associated with successful compliance (e.g., training samples comprising subject records, associated datasets, and outputs obtained or derived from a plurality of subjects known to have successful compliance). Independent training samples may be associated with unsuccessful compliance (e.g., training samples comprising subject records, associated datasets, and outputs obtained or derived from a plurality of subjects known to have unsuccessful compliance).

The trained algorithm may be trained with at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The independent training samples may comprise a positive set of subject records associated with successful compliance and/or a negative set of subject records associated with unsuccessful compliance.

The trained algorithm may be trained with a first number of independent training samples associated with successful compliance. The first number of independent training samples associated with successful compliance may be no more than the second number of independent training samples associated with unsuccessful compliance. The first number of independent training samples associated with successful compliance may be equal to the second number of independent training samples associated with unsuccessful compliance. The first number of independent training samples associated with successful compliance may be greater than the second number of independent training samples associated with unsuccessful compliance.

The trained algorithm may be configured to generate the outputs with an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more; for at least about 5, at least about 10, at least about 15, at least about 20, at least about 25, at least about 30, at least about 35, at least about 40, at least about 45, at least about 50, at least about 100, at least about 150, at least about 200, at least about 250, at least about 300, at least about 350, at least about 400, at least about 450, or at least about 500 independent training samples. The accuracy of generating the outputs by the trained algorithm may be calculated as the percentage of independent test samples that are correctly identified or classified as being likely to be converted.

The trained algorithm may be configured to generate the outputs with a positive predictive value (PPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The PPV of generating the outputs using the trained algorithm may be calculated as the percentage of subjects identified or classified as being likely successful conversions that correspond to subjects that truly were successfully converted.

The trained algorithm may be configured to generate the outputs with a negative predictive value (NPV) of at least about 5%, at least about 10%, at least about 15%, at least about 20%, at least about 25%, at least about 30%, at least about 35%, at least about 40%, at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or more. The NPV of generating the outputs using the trained algorithm may be calculated as the percentage of subjects identified or classified as being unlikely successful conversions that correspond to subjects that truly were not successfully converted.

The trained algorithm may be adjusted or tuned to improve one or more of the performance, accuracy, PPV, and/or NPV of generating the outputs. The trained algorithm may be adjusted or tuned by adjusting parameters of the trained algorithm (e.g., a set of cutoff values used to classify subject records as described elsewhere herein, or parameters or weights of a neural network). The trained algorithm may be adjusted or tuned continuously during the training process or after the training process has completed.

After the trained algorithm is initially trained, a subset of the inputs may be identified as most influential or most important to be included for making high-quality classifications. For example, a subset of the plurality of features of the subject records may be identified as most influential or most important to be included for making high-quality classifications or identifications. The plurality of features or a subset thereof may be ranked based on classification metrics indicative of each individual feature's influence or importance toward making high-quality classifications or identifications. Such metrics may be used to reduce, in some cases significantly, the number of input variables (e.g., predictor variables) that may be used to train the trained algorithm to a desired performance level (e.g., based on a desired minimum accuracy, PPV, NPV, or a combination thereof). For example, if training the trained algorithm with a plurality comprising several dozen or hundreds of input variables in the trained algorithm results in an accuracy of classification of more than 99%, then training the trained algorithm instead with only a selected subset of no more than about 5, no more than about 10, no more than about 15, no more than about 20, no more than about 25, no more than about 30, no more than about 35, no more than about 40, no more than about 45, no more than about 50, or no more than about 100 such most influential or most important input variables among the plurality can yield decreased but still acceptable accuracy of classification (e.g., at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%). The subset may be selected by rank-ordering the entire plurality of input variables and selecting a pre-determined number (e.g., no more than about 5, no more than about 10, no more than about 15, no more than about 20, no more than about 25, no more than about 30, no more than about 35, no more than about 40, no more than about 45, no more than about 50, or no more than about 100) of input variables with the best classification metrics.

### Computer Systems

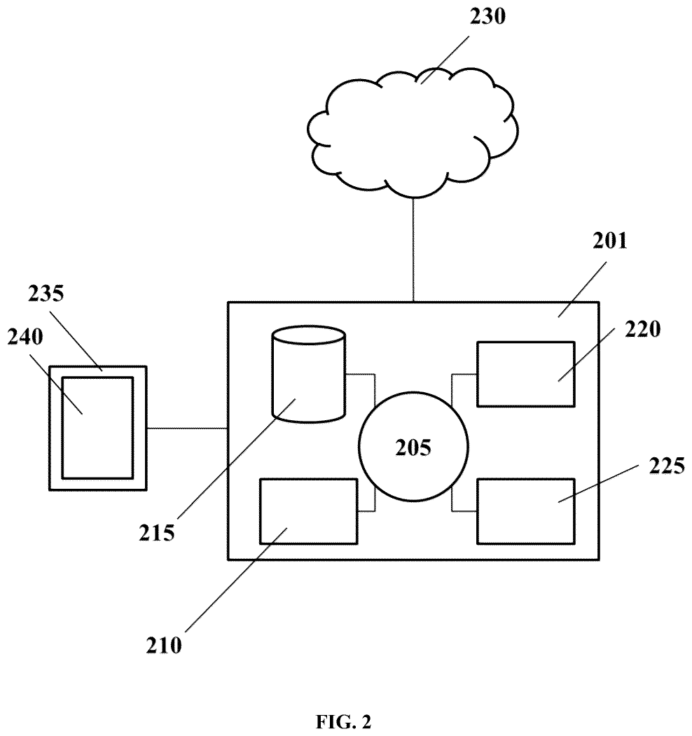

The present disclosure provides computer systems that are programmed to implement methods of the disclosure. FIG. 2 shows a computer system 201 that is programmed or otherwise configured to, for example, train and test a trained algorithm; retrieve subject records from a computer database; and process subject records using a trained machine learning algorithm to generate a subset of subject records.

The computer system 201 can regulate various aspects of analysis, calculation, and generation of the present disclosure, such as, for example, training and testing a trained algorithm; retrieving subject records from a computer database; and processing subject records using a trained machine learning algorithm to generate a subset of subject records. The computer system 201 can be an electronic device of a user or a computer system that is remotely located with respect to the electronic device. The electronic device can be a mobile electronic device.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage and/or electronic display adapters. The memory 210, storage unit 215, interface 220 and peripheral devices 225 are in communication with the CPU 205 through a communication bus (solid lines), such as a motherboard. The storage unit 215 can be a data storage unit (or data repository) for storing data. The computer system 201 can be operatively coupled to a computer network (“network”) 230 with the aid of the communication interface 220. The network 230 can be the Internet, an internet and/or extranet, or an intranet and/or extranet that is in communication with the Internet.

The network 230 in some cases is a telecommunication and/or data network. The network 230 can include one or more computer servers, which can enable distributed computing, such as cloud computing. For example, one or more computer servers may enable cloud computing over the network 230 (“the cloud”) to perform various aspects of analysis, calculation, and generation of the present disclosure, such as, for example, training and testing a trained algorithm; retrieving subject records from a computer database; and processing subject records using a trained machine learning algorithm to generate a subset of subject records. Such cloud computing may be provided by cloud computing platforms such as, for example, Amazon Web Services (AWS), Microsoft Azure, Google Cloud Platform, and IBM cloud. The network 230, in some cases with the aid of the computer system 201, can implement a peer-to-peer network, which may enable devices coupled to the computer system 201 to behave as a client or a server.

The CPU 205 may comprise one or more computer processors and/or one or more graphics processing units (GPUs). The CPU 205 can execute a sequence of machine-readable instructions, which can be embodied in a program or software. The instructions may be stored in a memory location, such as the memory 210. The instructions can be directed to the CPU 205, which can subsequently program or otherwise configure the CPU 205 to implement methods of the present disclosure. Examples of operations performed by the CPU 205 can include fetch, decode, execute, and writeback.

The CPU 205 can be part of a circuit, such as an integrated circuit. One or more other components of the system 201 can be included in the circuit. In some cases, the circuit is an application specific integrated circuit (ASIC).

The storage unit 215 can store files, such as drivers, libraries and saved programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet.

The computer system 201 can communicate with one or more remote computer systems through the network 230. For instance, the computer system 201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display 235 that comprises a user interface (UI) 240 for providing, for example, a visual display indicative of training and testing of a trained algorithm; a visual display of a set of subjects and/or subject records; a visual display of a medical image. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 205. The algorithm can, for example, train and test a trained algorithm; retrieve subject records from a computer database; and process subject records using a trained machine learning algorithm to generate a subset of subject records.

### EXAMPLES

### Example 1—Customer Relationship Management System

Using systems and methods of the present disclosure, a customer relationship management (CRM) system is configured as follows. An Anticipatory Consumer Technology (ACT) platform is configured as part of a screening mammography compliance program in which outreach is performed for subjects (e.g., patients) of radiology clinic groups. The ACT platform is used to increase mammogram volumes and compliance at partner clinics using various outreach programs. Parties involved may include the patients and call center agents performing outreach to the patients.

The ACT platform may enable spreadsheet-based outreach to patients, such as by part-time clinic staff or dedicated call center agents (using sales and marketing approaches). The ACT platform may be deployed to work with existing infrastructure (e.g., phone system, EHR/RIS system, booking and scheduling system). The ACT platform may be integrated into client workflows, and may take into account call center metrics and agent performance (e.g., booked to reached ratio, bookings per day, time per call, calls per day). The ACT platform may scale outreach efforts to optimize caller efficiencies. The ACT platform may include automated quality assurance, such as natural language processing (NLP) based QA, language-based outreach, AI-based outreach, and outreach outside of screening mammograms (e.g., any other diagnostic test, screening test, initial clinical procedure, or follow-up clinical procedure).

The ACT platform may address inefficiencies in call center outreach based on various challenges to ensuring patient compliance, such as reasons why patients do not return for follow-up screening or clinical procedures, and how their behavior can be changed. The ACT platform may measure metrics such as what resonates with patients (e.g., objection handling), why certain patients are more likely to be converted (e.g., due to sales skills), and overall performance in terms of number, percentage, or rate of non-compliant patient conversions. The ACT platform may enable QA-driven training insights, such as large amounts of call recording data, call center metrics, and agent performance metrics (e.g., booked to reached ratio, bookings per day, time per call, calls per day) that enables call center agents to learn and improve from mistakes.

The ACT platform may enable calling scripts using keywords and phrases to enable seamless transition to a new AI or QA based script. Standardized scripts may help agents offer accurate information to patients, speed up booking time and increasing daily conversion rates. The ACT platform may enable new client onboarding to be performed remotely. The ACT platform may enable productizing of education to replicate success and consistency.

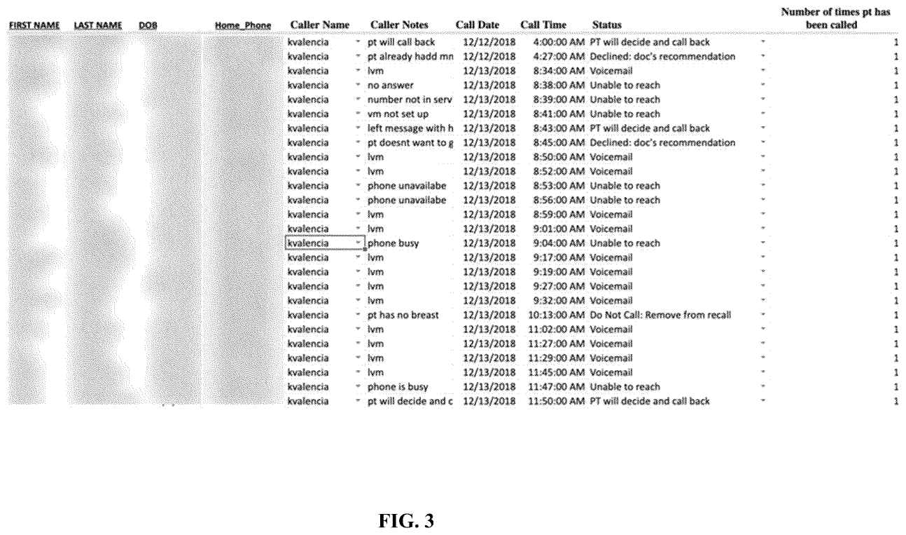

FIG. 3 shows an example of a spreadsheet-based outreach performed using the ACT CRM system. Worklists are pulled from an existing RIS system. Caller expectations may be set at a pre-determined level (e.g., 60 calls per day). This outreach may be a manual process.

FIG. 4 shows an example of an automated outreach performed using the ACT CRM system. Automated worklists may be generated by the ACT system to implement a nine-stage outreach program (e.g., 3 calls, and the rest texts). The ACT system may enable granular patient statuses to track customer journey, and scaling from 10 clinics to 350+ clinics. Caller expectations may be improved to a higher level (e.g., 120 calls per day). The ACT system may enable a point-based incentive program based on number of bookings. The ACT system may provide call center agents with a user-friendly tool to perform outreach.

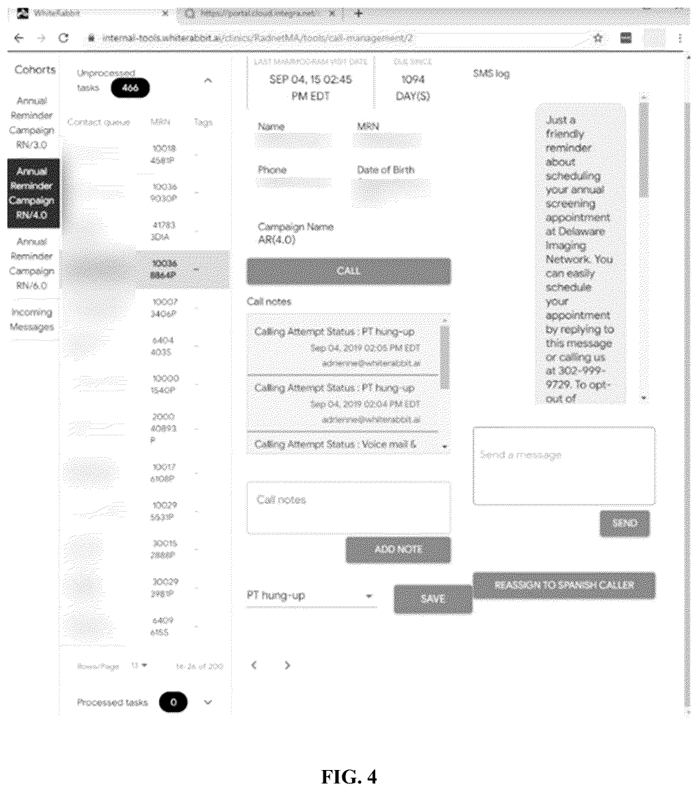

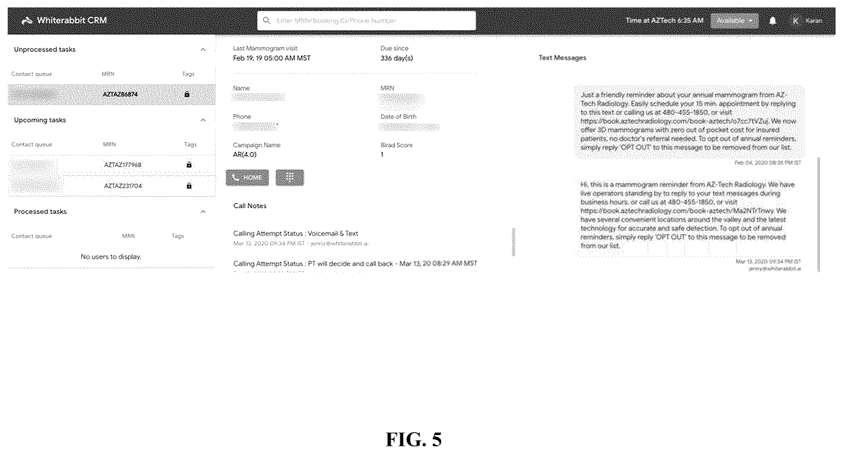

FIG. 5 shows an example of an automated outreach performed using the ACT CRM system. The ACT system may enable remote work for employee safety or convenience. The ACT system may enable call functionality. The ACT system may enable prioritization of high-converting patients (e.g., with higher AI/ML model compliance scores). The ACT system may enable agent tracking visibility. The ACT system may enable improved caller expectations (e.g., 120 calls per day) and/or improved conversion ratio (e.g., 50% or better). The ACT system may enable optimized or maximized caller efficiency to provide higher performance outreach with a fewer number of call center agents.

The ACT system may enable machine learning model integration, and may be adapted to any clinical procedure (e.g., beyond screening mammograms). The ACT system may enable a self-serving portal for users to opt out sets of patients, add exclusions based on providers, view reporting and metrics, automate the account manager role, etc.

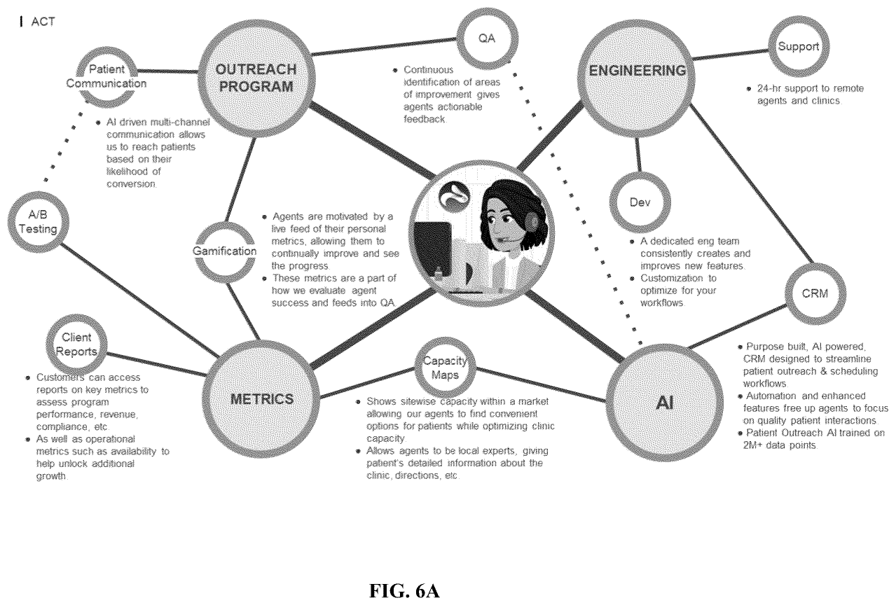

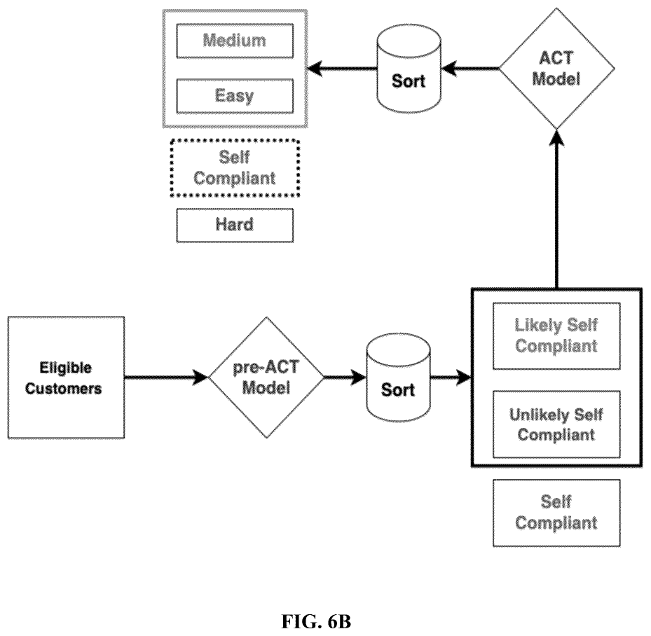

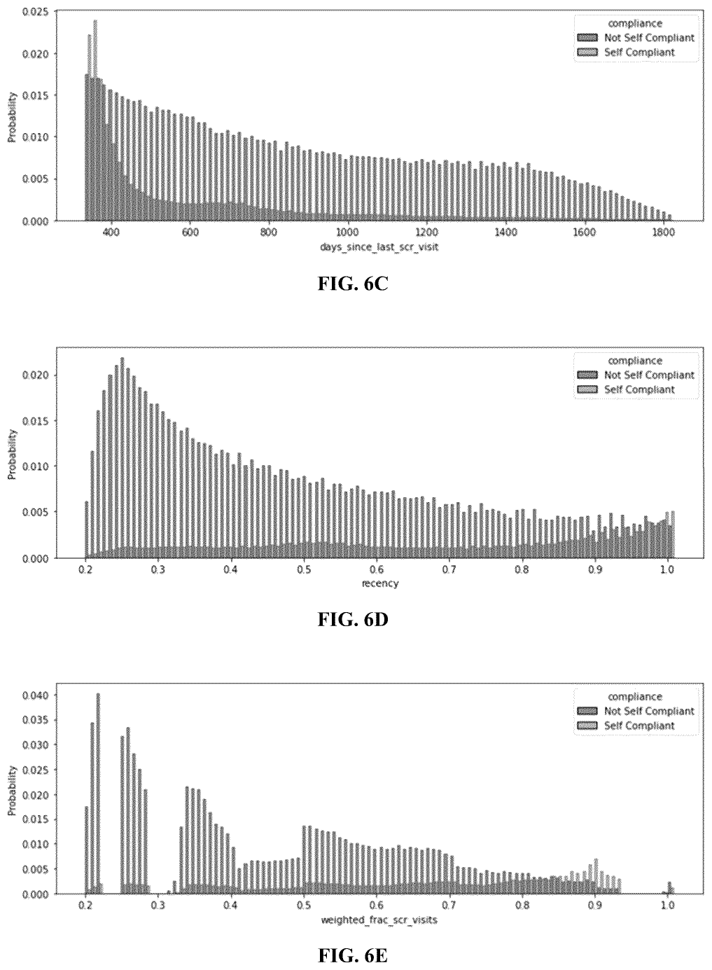

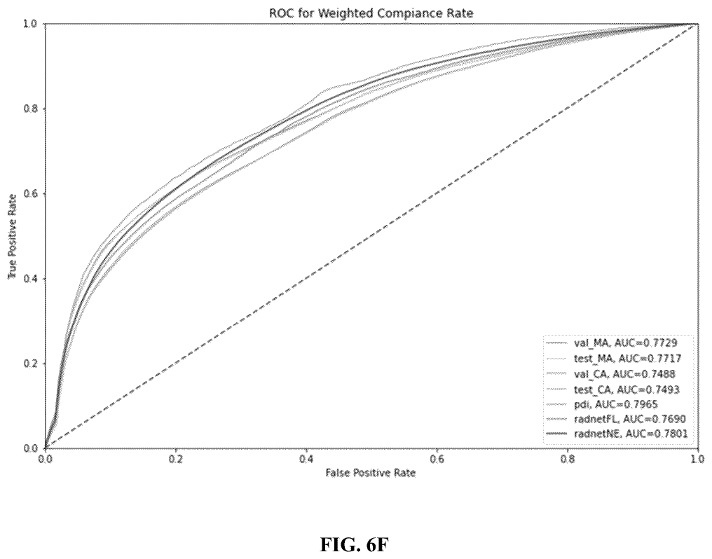

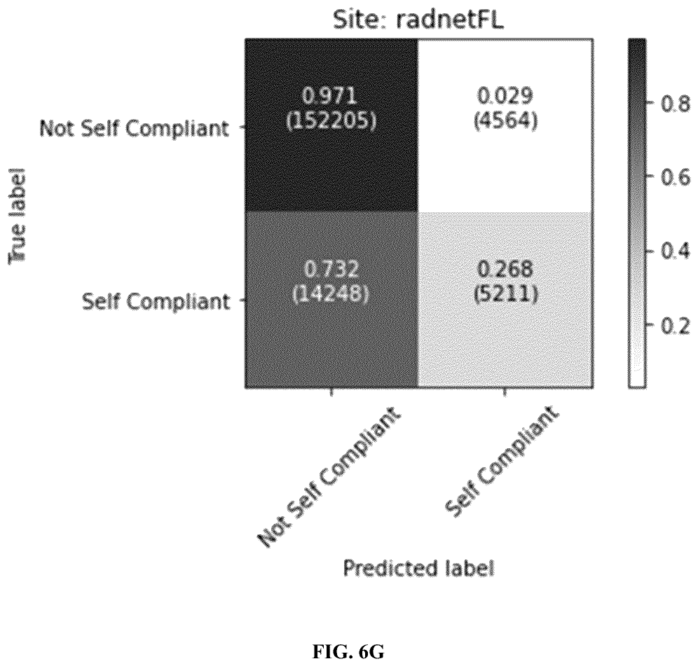

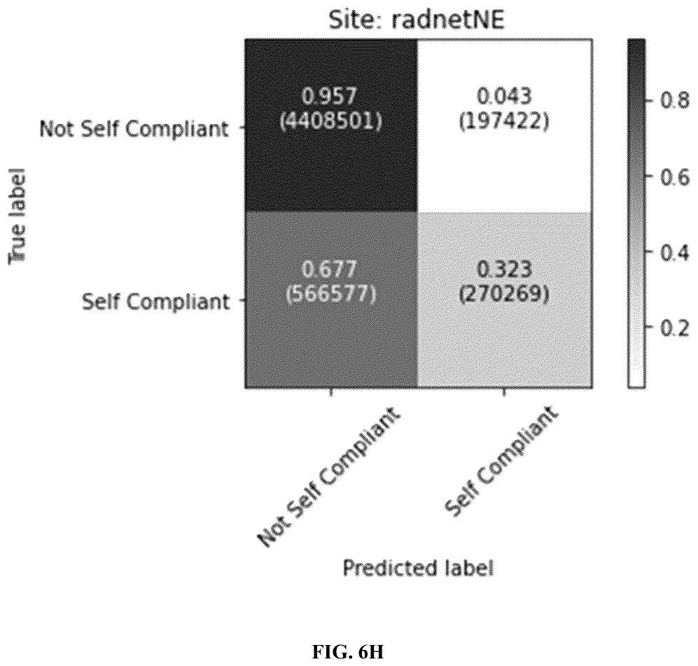

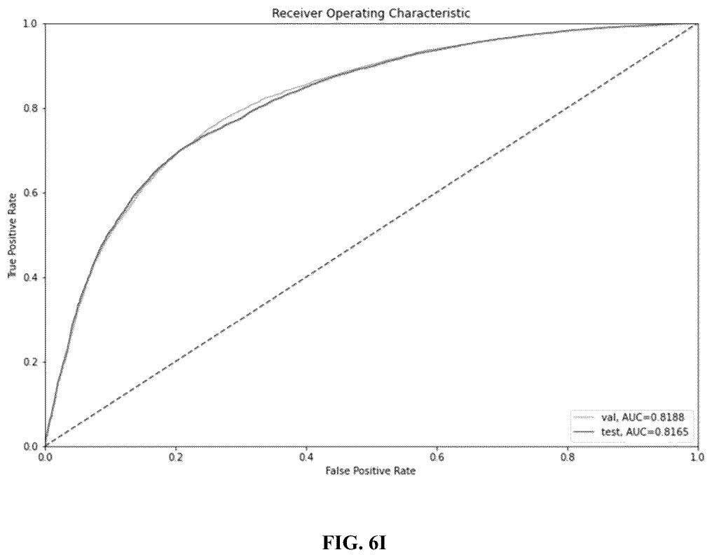

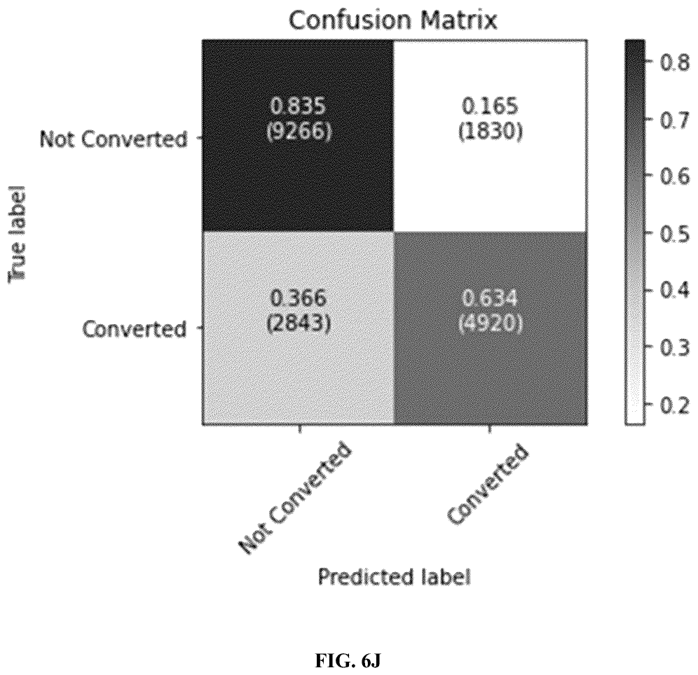

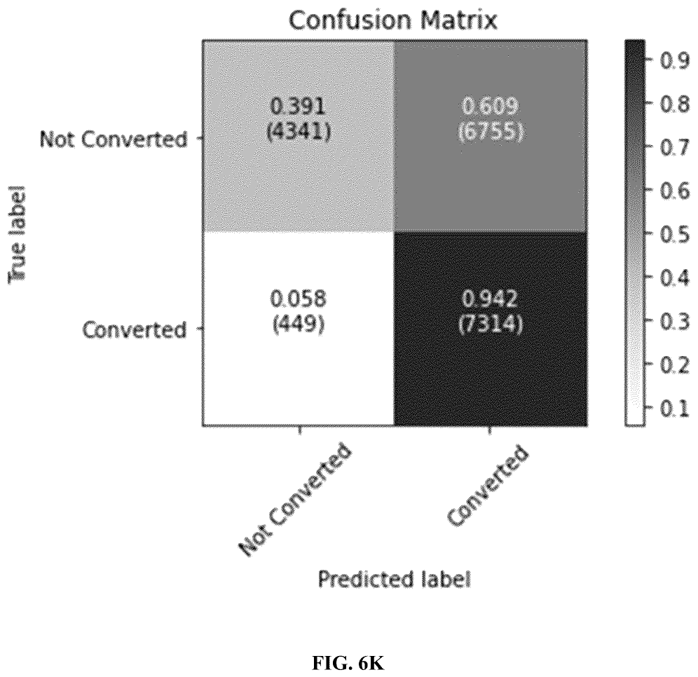

FIG. 6A shows an example of the integration of various aspects into the ACT system, including an outreach program, call center metrics, engineering, and AI/ML models.

### Example 2—Customer Relationship Management System

Using systems and methods of the present disclosure, a customer relationship management (CRM) system is configured as follows. The customer relationship management system is integrated into radiology information systems that enables development, execution, coordination, automation, and evaluation of staged outreach campaigns via multiple communication channels (e.g., texting, phone calls, and others) for a clinical procedure. The customer relationship management system uses a machine learning model to predict customer behavior for outreach campaigns.

The clinical procedure may be any periodic procedure where the CRM system is configured with the following features. Machine learning and/or data science may be applied to choose an optimal cadence, channel, content for each outreach attempt to each patient.

Further, learning and/or data science may be applied to select a subset of patients for stages or channels that have a high reward or reduced cost, such as: texting as opposed to phone calls which need a call-center; compliant patients versus non-compliant patients; and identifying underserved patients and communities. Alternatively, the machine learning system can de-select a subset of patients from being contacted through a high-cost channel/stage.

FIG. 6B shows an example of an ACT model pipeline. With ACT, callers contact patients to come in for a periodic exam. Machine learning can be used to optimize the set of patients to call and in what order by estimating the likelihood that patients will come in of their own accord and the likelihood that outreach will be effective in getting them to come in. Patients are first classified as “self-compliant” via a PRE-ACT model. This model is trained on the data from a time period when the ACT program was not active. This model gives a probability of self-compliance of a customer and we can now bin them as self-compliant, likely to be self-compliant, or unlikely to be self-compliant. A second model can then predict the likelihood of conversion via ACT. We can further optimize which customers are brought in the ACT program and in what order we perform outreach. In this design, callers prioritize patients that are medium and easy and leave the self-compliant and hard patients for later as any outreach intervention on them may not likely change their outcome.

FIG. 6C shows an example of a probability of compliance of patients based on their days since last screening visit. A simple way to identify self-compliant patients is to look at their number of days since the last screening mammogram visit. We may expect more self-compliant patients to have a lower number of days since the last screening visit. As shown in FIG. 6C, as expected, we find a much heavier tail for the non-self-compliant patient as compared to the self-compliant ones. We define a patient as self-compliant if they make a booking within 120 days from the day they become eligible for a screening mammogram, e.g., 11+ months after a previous visit.

FIG. 6D shows an example of a probability of compliance of patients based on their recency. We define recency of a customer as reciprocal of the number of years since last screening visit. This makes this parameter bounded between 0 and 1. Similar to the days since last screening visit, we find a heavier tail for the non-self-compliant customers.

FIG. 6E shows an example of a probability of compliance of patients based on their weighted compliance rate. We define the compliance rate of a patient as a fraction of the number of screening visits in the last 5 years and the number of maximum possible visits since their first visit in the last five years, assuming a screening interval of 12 months. The weighted compliance rate can then be defined as square root of the product of recency and compliance rate. This parameter captures the more frequent as well the more recent patients. For example consider two patients, both have had 2 visits in the last 5 years out of a max of 5 possible visits. However, if one patient's last visit was more recent, she would be more likely to be compliant than the one whose last visit was much further back.

FIGS. 6F-6H show an ROC curve (FIG. 6F) and confusion matrices (FIGS. 6G-6H) for prediction of patient self-compliance based on weighted compliance rate.

FIGS. 6I-6K show an ROC curve (FIG. 6I) and confusion matrices (FIGS. 6J-6K) for prediction of whether a patient is likely to convert via the ACT CRM outreach.

Further, the ACT CRM system may use machine learning and/or data science to drive operational choices of allocating high-cost resources (such as call-center agents) across various categories (such as customer markets) optimizing for a goal, such as a sum of projected revenue per market. This optimization may employ the above predictive models to improve the choices.

Further, the ACT CRM system may use machine learning and/or data science to drive outreach cadence, channel, and/or content that aims to spatially shift patient populations. For example, if a site has a large population eligible for their next periodic follow-up, but is unable to serve such a large volume, the outreach encourages that population to go to nearby sites which have capacity.

Further, the ACT CRM system may use machine learning and/or data science to drive outreach cadence, channel, and/or content that aims to temporally shift patient populations. For example, if the periodic healthcare procedure exhibits high seasonal volume, e.g., October breast cancer awareness month, the outreach encourages that population to pull in or to defer their procedure to a less busy period.

### Example 3—Customer Relationship Management System

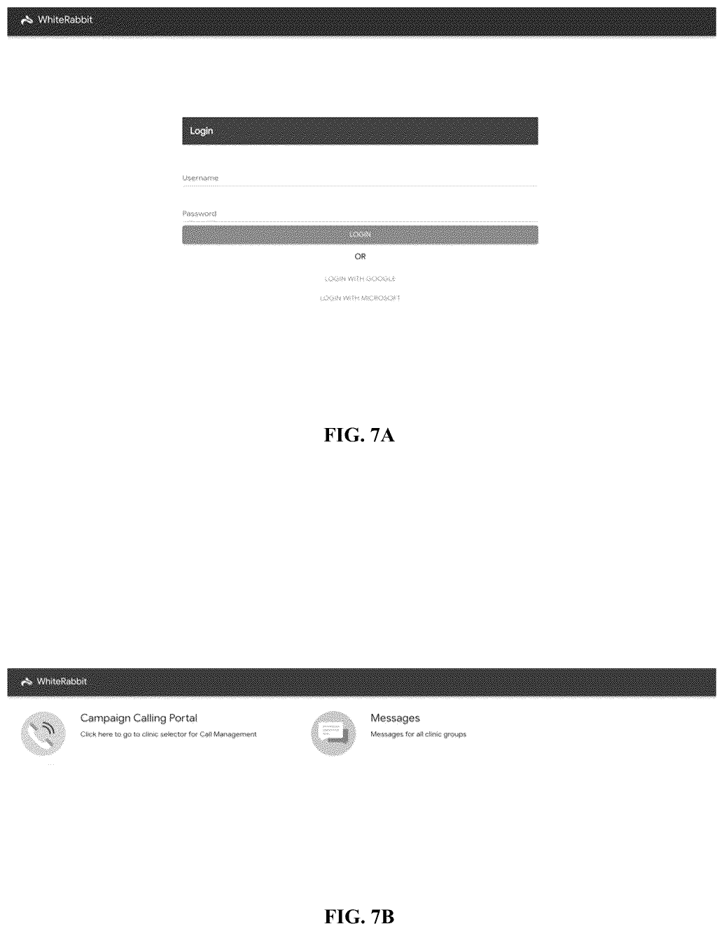

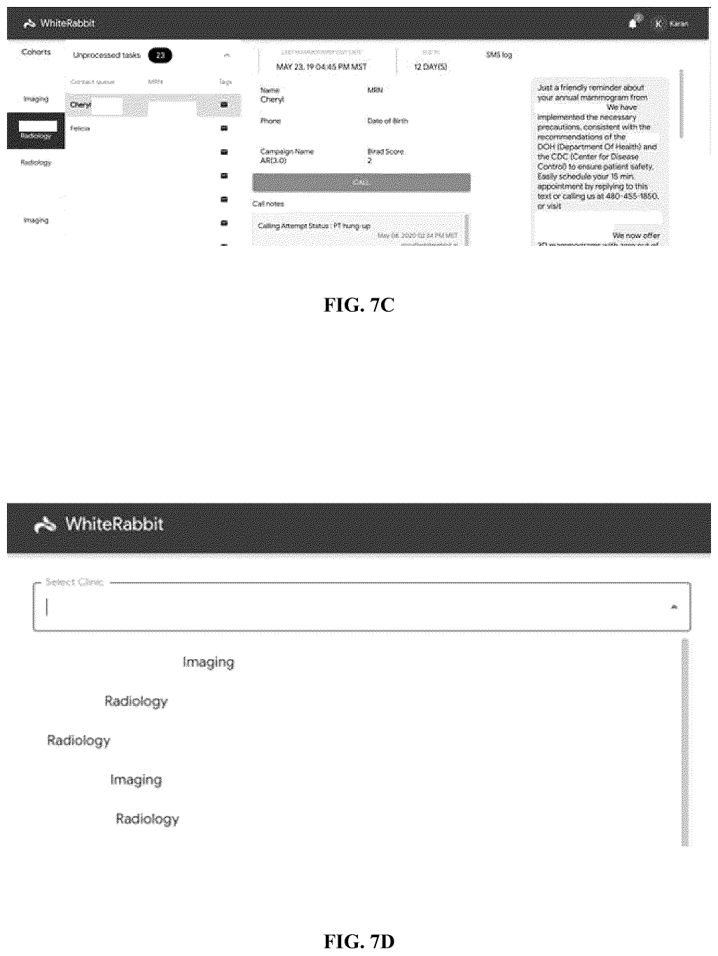

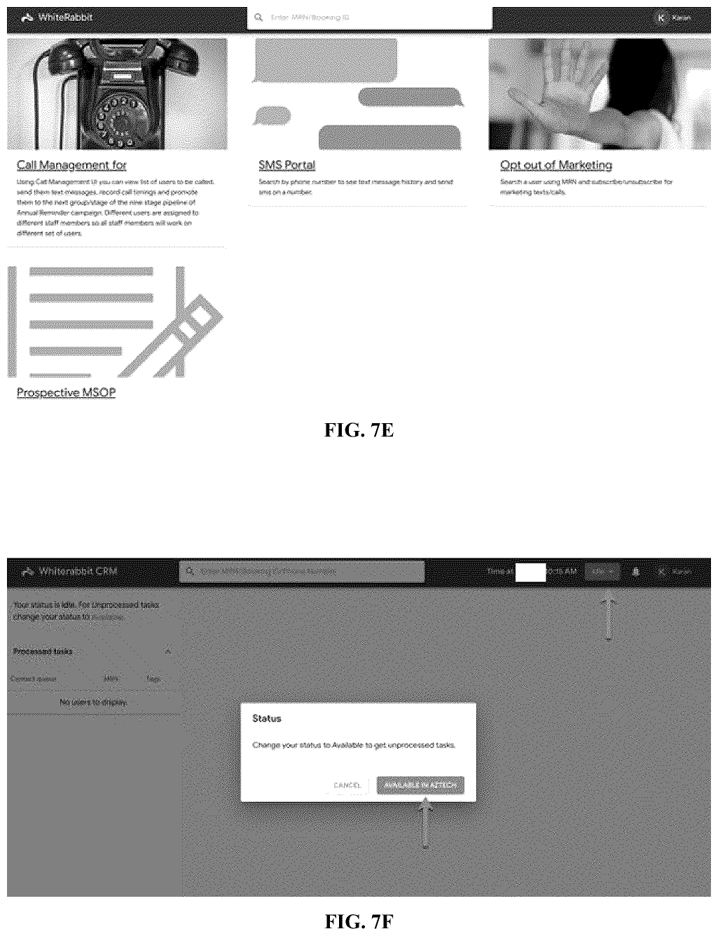

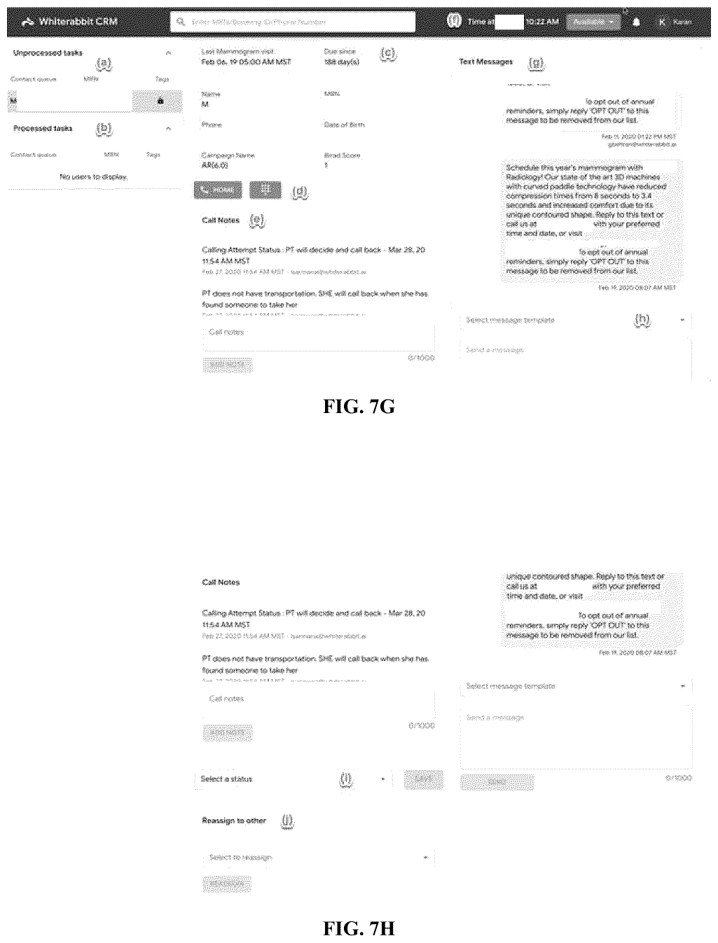

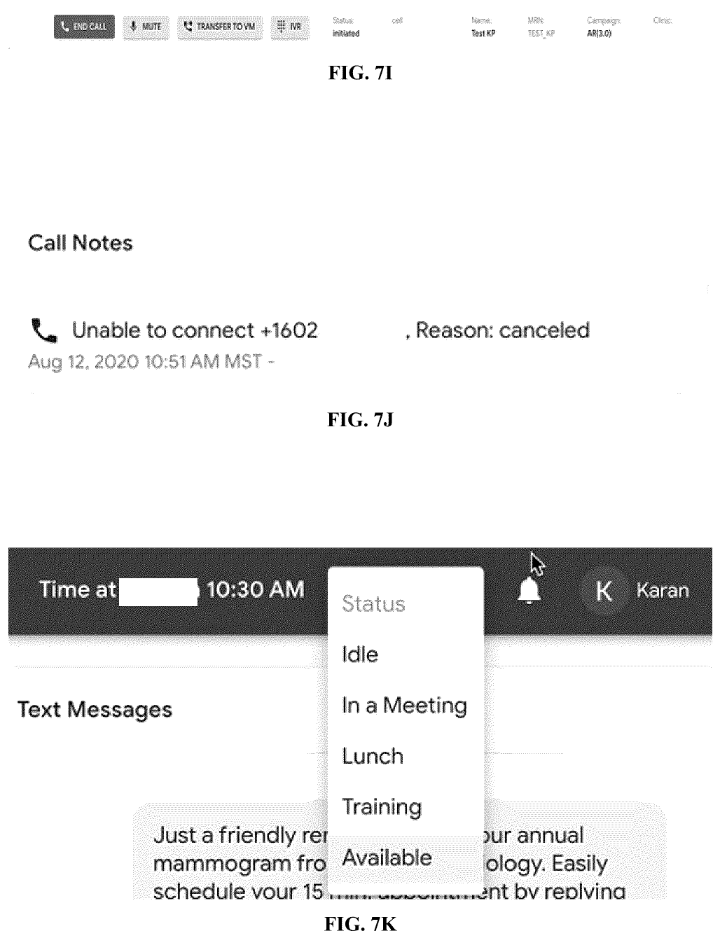

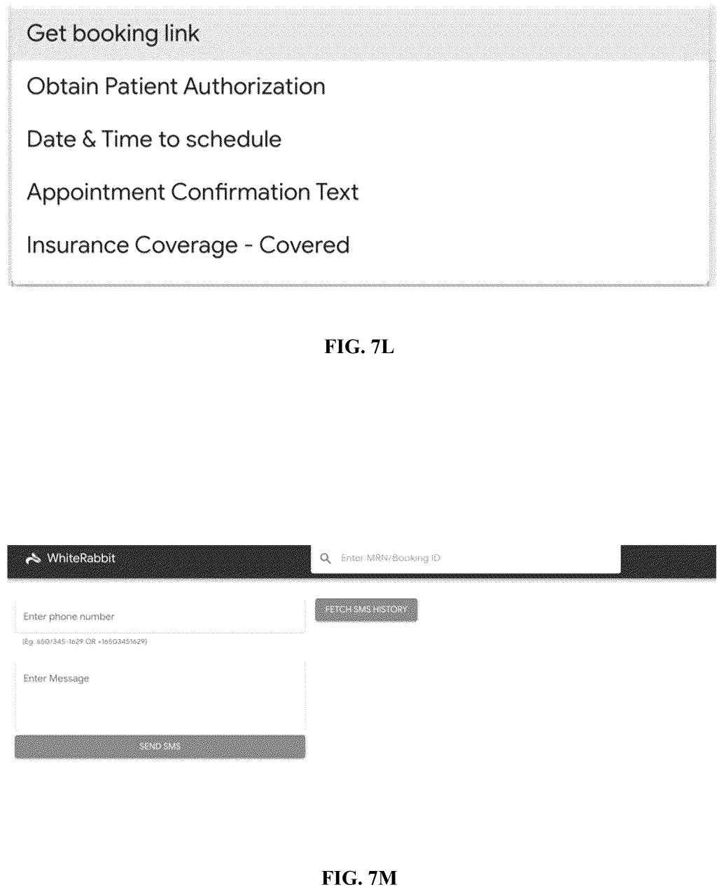

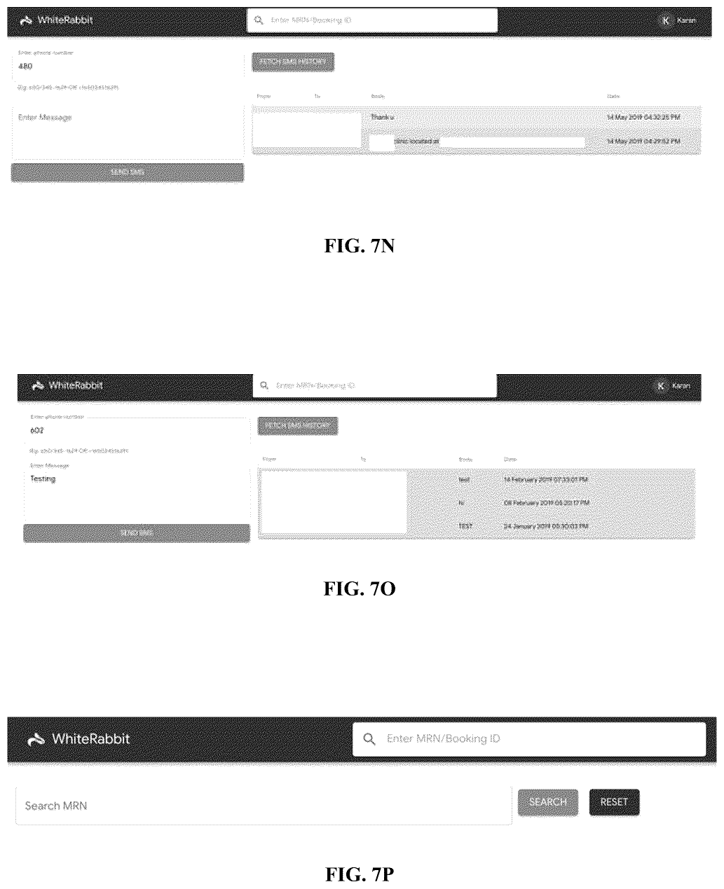

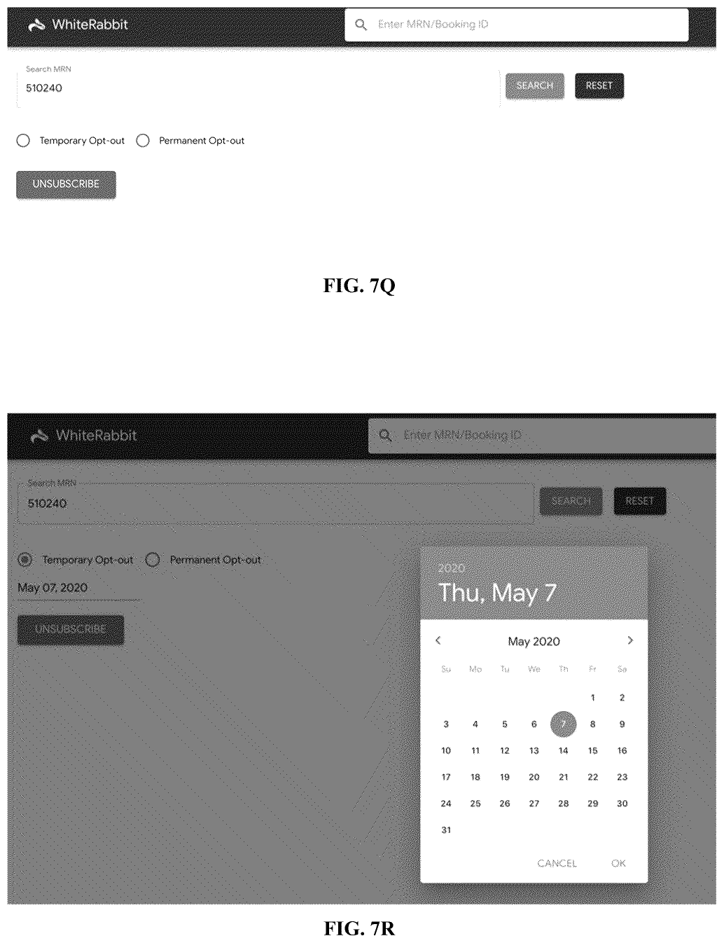

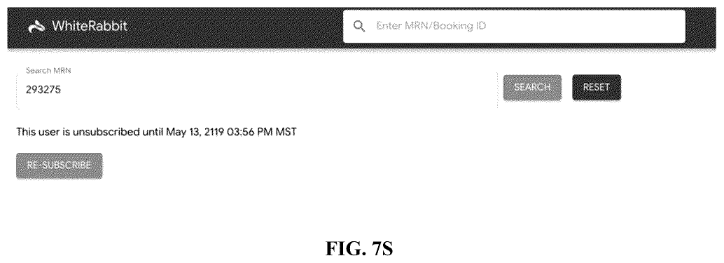

Using systems and methods of the present disclosure, a customer relationship management (CRM) system is configured using the following CRM system product specification. FIGS. 7A-7S show example screenshots of a user interface of the CRM system.

In order to access the CRM, each user needs to be granted access. The user needs to go to a provided website. Upon going to the website, a user setup screen pops up as shown in FIG. 7A.

Depending on which email domain the user's email is set up, the user selects “Login with Google” or “Login with Microsoft”. The user sees a message saying they are not associated with a clinic. At this point, the user needs to request access to the CRM with the market they need access to. Once access is granted, the user is able to log in using the same link.

The staging link can be used for testing and training purposes. The same process needs to be followed to get access (log in once, see error message, etc.).

The CRM provides user access to one or more tools. Upon logging into the CRM, the user is asked to choose a portal-Campaign Calling or Messages (as shown in FIG. 7B). The campaign calling is for agents who are assigned to make outbound calls. The messages portal is for agents who are designated to respond back to customers via texting.

Upon selecting the messages portal, the user is taken directly to the worklists which contain the markets they are assigned to (as shown in FIG. 7C).

Upon selecting the campaign calling portal, the user is asked to choose a clinic, and there is displayed a drop down menu after logging in (as shown in FIG. 7D).

After selecting the clinic under the calling campaign, a screen appears as shown in FIG. 7E. Within the CRM, there are four distinct tools. First, the Call Management (the corresponding clinic name appears) is the main tool used by the calling team. Second, the SMS portal is a standalone program that gives the user the ability to type in a free form text to send to a specific phone number. Third, the “Opt out of marketing” is to opt customers out of receiving messaging and calls for the outreach campaigns. Fourth, the prospective MSOP (Mammogram Screening Optimization Program) is for in-clinic conversion in which patients visiting for other exams are determined to be eligible for screening mammography and encouraged to get a mammogram. Further details of each tool and how to utilize them are provided below. A given user may or may not have access to each of these tools, depending on their role within the company or entity.

The call management tool is based on a 9 stage logic for the annual reminder campaign. The due date refers to the date the customer is due for their annual screening mammogram. The 9 stages include the following:
1) 30 days before the due date: the customer receives a letter from the Mammography Reporting Software (MRS). This can change based on the client but this is the typical first step.
2) 14 days before the due date: first automated text message reminder is sent out. This stage is also known as stage 2.
3) 7 days before the due date: first phone call made by the user. If the user selects voicemail and text, an automated text message is also left for the customer. This is known as stage 3 or annual reminder campaign 3.
4) Due date: second phone call made by the user. If the user selects voicemail and text, an automated text message is also left for the customer. This is known as stage 4 or annual reminder campaign 4.
5) 7 days overdue: second automated text message reminder, this is known as stage 5.
6) 14 days overdue: third and final phone call made by the user. If the user selects voicemail and text, an automated text message is also left for the customer. This is known as stage 6 or annual reminder campaign 6.
7) 30 days overdue: third automated text message reminder.
8) 60 days overdue: fourth automated text message reminder.
9) 90 days overdue: fifth automated text message reminder. This is the final outreach performed for that subject for the year. If the customer does not opt out, they return in the following year.

The call management workflow is provided as follows. A screen (as shown in FIG. 7F) is shown to the user after selecting Call Management. This is primarily used by users who are making calls to patients for the various mammography growth campaigns (annual reminder campaign, MSOP, incoming messages). Upon selecting the call management tool, users are taken to the screen and asked to become “available” in the respective market selected. In order to receive customers, an agent must be available.
(a) Upon becoming available, a customer is assigned to the agent (as shown in FIGS. 7G-7H). Depending on the permissions granted to the agent, they can receive a customer from different campaigns. Only one customer is assigned to an agent at a time. The agent must process this customer before another one is assigned. For example, for the annual reminder (AR), the nine stage outreach campaign is performed to remind customers about their annual mammogram. As another example, for the MSOP, the customers within these campaigns are females above the age of forty (groups can vary between clinics) who have not been to our clinic for a mammogram within a certain time frame (can vary between clinics) but have come to us for other radiology based exams (x-ray, CT, MRI's, etc.).
(b) Processed tasks show the user how many customers they have processed for that specific day.
(c) In this area, agents find the most recent screening mammogram of the customer along with their demographic information below. This information is pulled directly from the RIS system. Above this area, the user is able to search MRN & Booking ID (Booking ID is PDI only for online appointments.
(d) The call feature has been built into the internal CRM. If an agent clicks on the home button, the system begins dialing the home phone number. Agents can hover over the button to see what phone number they are dialing. The dial pad to the right of the buttons allows agents to manually input any numbers they wish to dial. Upon starting the call, a toolbar appears at the bottom of the page (as shown in FIG. 7I). The transfer to VM (voicemail) button should be used when leaving a voicemail message for the customer. Please be sure to hit the button after hearing the beep. The phone call automatically disconnects after that and the system leaves the voicemail. The IVR dial pad can be used to mitigate any robocalls protectors-you can input numbers after the call has begun.
(e) Upon completing a call, notes are automatically populated in the notes field (as shown in FIG. 7J).
(f) The bell feature is where notifications show up for the user (as shown in FIG. 7K). There are notifications for callback times that have been triggered. The user has the notification there until it has been handled by any user. In this area, also displayed is the time zone of the market selected and the status the agent is currently in. The statuses are shown in FIG. 7K.
(g) The SMS log area shows previous text message contact we have had with the customer and gives the user the capability to send text messages directly to the customer.
(h) The drop down menu has predefined text messages ready to send (as shown in FIG. 7L). For example, if an agent selects “Obtain Patient Authorization, the following text appears: “Hi, my name is Karan, I would be happy to help you schedule your appointment. Communication via text is not secure and we cannot ensure the confidentiality of information discussed here. In order to get scheduled via text please verify your identity by replying YES and include your First and Last Name. If not, I can schedule a time to have someone call you.” Certain predefined text messages require the agent to fill in additional information.
(i) Select a Status; In this area, the caller can select the status of the customer they called. The user is required to select a status before moving onto the next patient. Once the status is selected, the user must ensure they click save in order for the customer to be moved along in the campaign accordingly. Table 1 shows different CRM statuses, interpretation, scenarios, and customer flow of each status.

TABLE 1
CRM Statuses

| Status in UI | Interpretation | Scenarios | Customer flow |
|---|---|---|---|
| Voicemail & | The call went to voicemail & | 1. User left a voicemail | The customer will continue |
| Text | the user transferred the call to | 2. If unable to identify the | into the next stage of the |
| | the recorded voicemail. An | customer, the user is ok to | campaign they are in. |
| | automated text will be sent if | leave a VM | |
| | the phone number is textable. | | |
| Booked | The call connected & an | 1. Booked customer for | The customer will fall off the |
| | appointment was booked. | mammogram, if another | campaign list and return next |
| | | modality is also booked with | year in the annual reminder |
| | | the MG, enter into incentive | campaign. |
| | | sheet | |
| | | 2. Do not mark as booked if | |
| | | only scheduling other modality | |
| Declined: | The call connected & the | 1. Customers who are currently | The customer is removed |
| PT pregnant | customer told the caller that | pregnant | from the campaigns and re- |
| | they are pregnant, hence they | 2. This also applies for | enters in the following year. |
| | cannot have a mammogram. | breastfeeding customers. | |
| Declined: | The call connected & customer | 1. Customers insurance is not | The customer is removed |
| non- | informed the caller about the | in network with client group | from the campaigns and re- |
| contracted | change of insurance/preferred | | enters in the following year. |
| insurance | center. | | |
| Declined: | The call connected & the | 1. Referring doctor is | The customer is removed |
| doc's | customer told the caller that | recommending not to have it | from the campaigns and re- |
| recommend | their doctor has recommended | every year or not to have | enters in the following year. |
| ation | not to have MG or to have it | mammograms at all. | |
| | every two years. | 2. Customers referring | |
| | | provider does not want them to | |
| | | use our clinic | |
| Declined: | The call connected & the | 1. Customer moved away, the | The customer will be |
| moved away | customer has moved from our | user needs to input state in | removed from our campaigns |
| | service area. | notes section | & not appear in our |
| | | | campaigns in the following |
| | | | years. |
| Declined | The call connected and the | 1. Customer declined and did | The customer will be |
| | customer declined to schedule | not provide a reason | removed from our campaigns |
| | an appointment. | 2. Customer does not want to | and will appear in our system |
| | | get a mammogram done | in the following years. |
| PT will | The call connected and the | 1. Customer insists that they | The customer will continue |
| decide and | customer said they will decide | will call back - the user should | into the next stage of the |
| call back | and call back. | try to offer request callback | campaign they are in but they |
| | | instead. | will not arrive into the next |
| | | | campaign until 1 month later. |
| PT | The call connected and the | 1. Customer requested a call | The customer will appear in |
| requested | customer asked to call again at | back at a specific time or time | the notifications area once the |
| a callback | a later time/date. Input time | frame | inputted call back time is |
| (time-input) | and customer goes into call | 2. Customer is sick and caller | triggered. |
| | back campaign. | believes they can book them at | |
| | | a later date | |
| Callback: | The call connected and we | 1. The customer requested a | The user will be prompted to |
| slot not | could not book as no preferred | specific time and that time slot | select a callback time for a |
| available | slots were available. This | was not available | notification to appear in order |
| | might also occur if US and | | to call the customer again. |
| | MG are both needed. Set up a | | |
| | callback time with the | | |
| | customer to offer appts. | | |
| PT hung-up | The call was connected and the | 1. The customer ended the call | The customer continues in |
| | customer hung up. | on purpose | campaigns according to |
| | | 2. The customer didn't mean to | schedule after receiving an |
| | | end the call and possibly could | automated text message. |
| | | be in a bad connection area | |
| Do Not Call: | The call connected and the | 1. Customer says not to call | The customer will be |
| Customer | customer asked not to be | them back or to stop reaching | removed from all future |
| opted out | contacted again. The user will | out to them (they will be | communications and not re- |
| (DND) | need to opt out the patient. | permanently opted out) | appear in the campaigns. |
| Do Not Call: | Based on previous reports, do | 1. Based off research before | The customer is set to do not |
| Call not | not contact the customer. | the call, the customer is not | disturb and will |
| required | These are for BI-RADS 0, 3, 4, 5 | eligible for a screening | be removed from all |
| based on | or 6. Please email the breast | mammogram. User should | campaigns permanently. |
| history | coordinators if applicable to | leave a note of the bi-rad score | |
| | your market. | | |
| Do Not Call: | Based on previous report or | 1. Customer informs us that | The customer is set to do not |
| Has Cancer | during a call made, the | they have breast cancer or had | disturb and will |
| | customer informs us that they | breast cancer in the past | be removed from all |
| | have cancer or it is within their | | campaigns permanently. |
| | previous reports; therefore, we | | |
| | did not contact them. | | |
| Ineligible: | The call connected and the | 1. Customer has had a | The customer will re-enter the |
| PT had MG | customer mentioned that they | screening mammogram in the | UI as a notification at the |
| in past 12 | have had a mammogram | past 12 months at a different | selected time that the user |
| months | within the past 11 months. | clinic | entered. |
| PT already | Caller skipped calling, cases | 1. The customer is already | The customer is removed |
| booked at | where the customer entry is | booked at the time of the call | from the CRM and entered |
| time of call | wrongly present in the CRM as | within our clinic groups | into funnel next year |
| | customer had booked an | | based on their completed mg |
| | appointment with us very | | date. |
| | recently. | | |
| Unable to | Caller tried reaching customer, | 1. There is a busy tone | The customer continues in |
| reach | the number was unreachable. | 2. The caller reached someone | campaigns according to |
| | | other than customer (i.e. family | schedule and receive a text |
| | | member), leave a note in CRM | message. |
| | | 3. VM is full or not set up | |
| | | 4. Continuous ringing and | |
| | | unable to leave voicemail | |
| Phone | There was an automated | 1. The call won't connect | The customer is dropped from |
| number | message stating the phone | because the number is | the campaigns |
| invalid | number is no longer in | disconnected or not in service | and will reappear in the |
| | service/active. | etc. | following year. |
| | | 2. If the VM recording is | |
| | | clearly not the customer | |
| Pt has order | The user sees a screening | 1. The caller should leave a | The customer gets placed |
| pending | mammogram order within the | note on when order is received | back into the following stage |
| | RIS system already and | in the RIS system | in the campaign six months |
| | therefore, does not make an | | later. |
| | outreach attempt. | | |

If a user selects the SMS portal tool, a screen as shown in FIG. 7M appears. The SMS portal can be used for multiple functions. The first function is to review SMS history between users and the patient (automated or manually entered). The user has to input the phone number associated with the patient and select fetch SMS history (as shown in FIG. 7N). The second functionality is to send a message to a phone number. This use case occurs if the SMS function is not working for any reason under the Call Management or if the patient is requesting a text to a specific number. The user inputs the phone number and then enters in the message they wish to send. After reviewing the message, the user clicks send SMS and the text message sends (as shown in FIG. 7O).

Opt out of Marketing functionality is used to opt customers out of our marketing efforts either permanently or temporarily. As part of the nine stage annual reminder campaign, text messages are sent to the patient. Some customers request to be opted out of our campaigns. We specifically use the words “OPT OUT” in our text messaging. If a customer responds with any of the below keywords, they are automatically opted out of marketing and marked as DND within the CRM (they are case insensitive—e.g., STOP and stop are both opted out automatically): Stop, Stopall, Unsubscribe, Cancel, End, Quit, Opt Out, and Opt-out. For these particular patients, the user does not need to do anything further. It is very important to remember that it has to be these exact words. If a patient responds with “please stop”, they do not automatically get opted out. If a patient misspells a word from the list above, such as “unsubscribe”, they are not opted out.

For the other responses, the user needs to select the opt out of marketing tool and a screen appears as shown in FIG. 7P. The user needs to input the MRN associated with the patient in order to bring up a screen as shown in FIG. 7Q. Depending on the customer's request, the user is able to temporarily opt the customer out or permanently opt them out. For the temporary opt out, the user has a calendar pop up to enter a date to opt out the customer until (as shown in FIG. 7R). If the customer requests a permanent opt out, the user enters in the MRN and selects the permanent opt out and then selects unsubscribe. FIG. 7S shows an example of a customer who has been permanently opted out. Please also note in this screenshot, if you inadvertently DND a customer, you are able to re-subscribe them.

### Example 4—Customer Relationship Management System

Using systems and methods of the present disclosure, an ACT customer relationship management (CRM) system is configured as follows. A machine learning model is integrated into the CRM system to predict a quantity or characteristic of a subject (e.g., patient), and then take an action on that patient (e.g., perform outreach to that patient to receive a clinical procedure) or take an action on a person or practice affecting that patient (e.g., instruct a physician's office to schedule a clinical procedure for that patient). The machine learning model then optimizes and improves compliance rates with clinical procedures (e.g., receiving routine screening exams).

As an example, the ML model is applied to improve compliance rates with mammography screening exams. The ML model also enables additional functionalities in addition to driving compliance. Using data from the RIS/MIS/EMR, the ACT platform generates a behavioral conversion probability score that factors in a multitude of patient information. For example, the patient may include, but is not limited to: age, average income in zip code, home state, distance to clinic, total visits in past 3 years, screening mammograms in past 3 years, months since last screening mammogram, months since last non-screening mammogram, months since last other-modality exam, current time to day or day of week, and previous outreach attempts (e.g., texts, e-mails, calls, app pushes) and outcomes (e.g., delivered/undelivered, connected/voice mail, and declined/booked).

The model also factors in the probability of conversion based on outreach type, time of day, and outreach content, to programmatically select when and how to reach out to the patient. The ML model dynamically factors in failed outreach attempts and associated meta-data for subsequent attempts as patients move through subsequent stages of the ACT outreach campaign. If the AI selects SMS then one of the predefined text messages is sent to the patient at the time they are most likely to convert as determined by the ML model.

If the patient does not respond to the SMS then the ML model may decide that a voice call is the best follow up and populates the patient's information at the top of the call queue in the CRM at the appropriate time such that the next available agent attempts to reach that patient.

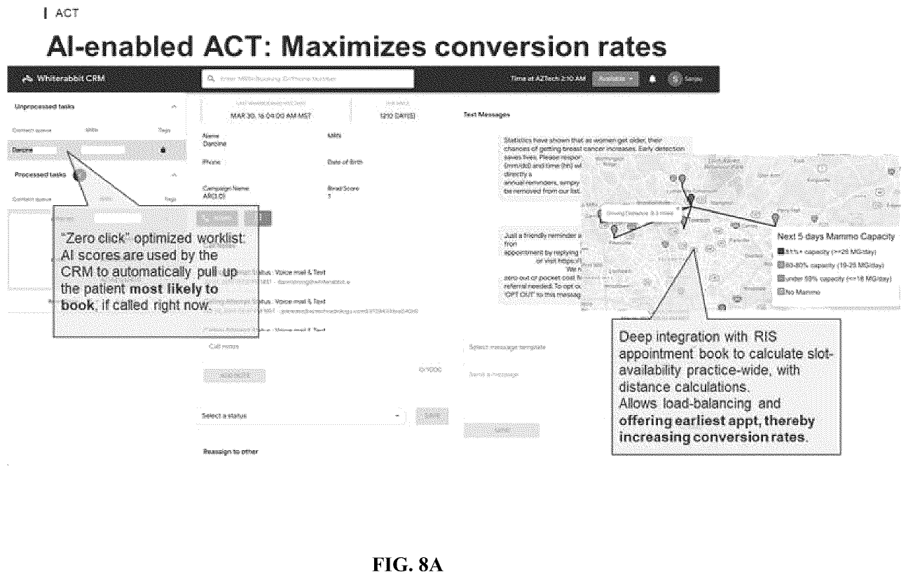

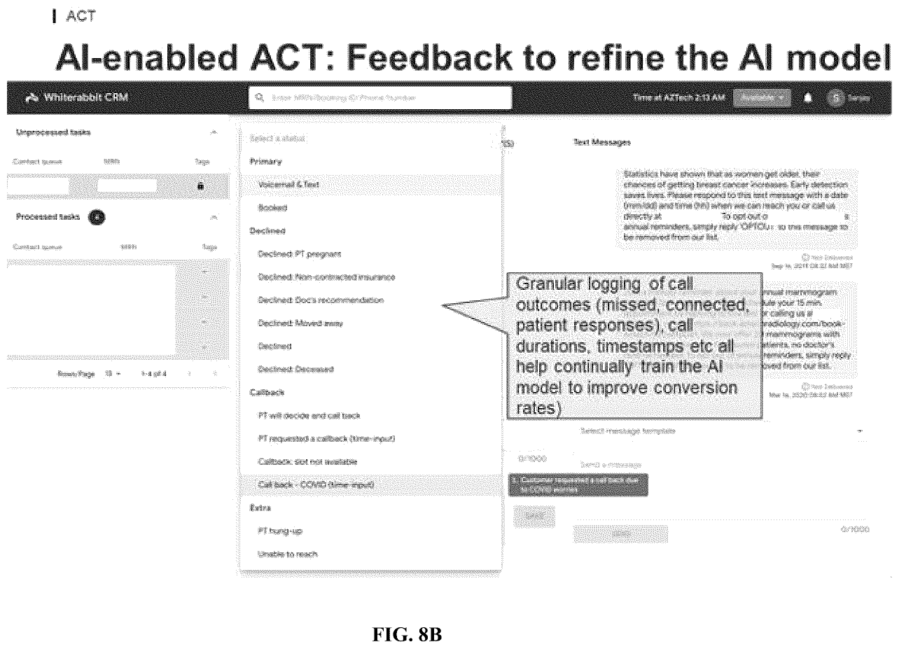

FIGS. 8A-8B shows an example of the ACT CRM system applied to mammography.

Further, the CRM system enables TAM (total addressable market) analysis to be performed based on prior visit history and modeling conversion rate. Automated models may be used to direct traffic to different providers via the CRM system based on various criteria.

As shown in FIG. 8A, the ML model suggests to patients and schedulers alike optimal locations of service based on a number of factors including but not limited to: Location of last visit; % capacity of site of last visit; Next available appointment at nearest location to site of last visit; Next appointment at site closest to addresses on file; Next available appointment at the same time as last visit; % capacity of proximal sites based on information on file. These suggestions are displayed visually to agents using the ACT CRM. These suggestions are displayed directly to patients booking using the web booking link via the site selection dropdown through dynamic sorting. These tools are especially useful in areas of high population density where multiple locations may be relevantly proximal.

Further, automated models may be used to move TAM, such as from capacity constrained months to less busy months to shift the peak load toward greater uniformity across the year. The machine learning load balancing model helps distribute seasonally skewed exam volume over the entire year. This reduces the duration and magnitude of high operational burden caused by high volume during certain parts of the year and can help unlock more consistent capacity throughout the year reducing the need for additional modality units and staff.

Further, the CRM system enables estimating patient convertibility and/or actions and applying the estimated patient convertibility and/or actions to workforce optimization. Depending on estimated potential of a given market, the CRM system suggests workforce allocation across the total pool of markets, such as by taking into market factors (e.g. capacity, last week's volume, no-show rate, inclement weather, etc.) and account user level characteristics. These characteristics may include but are not limited to: permissions, training, languages spoken, performance, rate of work, success rate, adaptability, customer service level, oral proficiency, written proficiency, etc.

Further, the CRM system enables estimating patient convertibility and/or actions, and applying the estimated patient convertibility and/or actions to scheduling and testing capacity optimization. Further, the CRM system enables estimating patient convertibility and/or actions, and applying the estimated patient convertibility and/or actions to optimized gathering of A/B testing data.

### Example 5—Customer Relationship Management System

Using systems and methods of the present disclosure, an ACT customer relationship management (CRM) system is configured as follows. The CRM system enables prediction of eligibility for medical tests and treatments. For example, retrospective MSOP campaigns (e.g., ultrasound and DEXA scans) may be performed using the CRM system. As the ACT CRM ingests comprehensive appointment data and patient data, the ML model is able to suggest additional exams patients may be due for based on their visit history that are additional to patients' initial reasons for visiting. This can be done both in a real time or prospective manner for patients who are either in the process of scheduling or have upcoming appointments. Alternatively, this can also be done in a retrospective manner for patients whose exams are already completed.

The ACT CRM automatically populates eligible patients based on the ML model's selected method of outreach into the appropriate outbound channel to maximize the likelihood of contact with the patients.

Further, the CRM system enables predictive eligibility for medical tests and treatments to be determined using targeting (e.g., AdWords). When the CRM system ingests comprehensive appointment data and patient data including patient data pertaining to treatments and prescribed pharmaceuticals, the ML model is able to suggest additional courses of treatment that may have either been overlooked, or predictively suggest follow-up treatments based on historical prescription regimen and medical prognosis and pathology extracted from physician's reports on file.

As an illustrative example, suppose a patient is diagnosed with cancer and prescribed a series of tests to determine the cancer genotype in order to enable personalized precision medicine. The ML model may suggest additional tests that were not initially ordered to provide a more comprehensive picture to the oncologist. The ML model may look at the results of the test that were received from the referring physician and recommend supplemental tests that were overlooked during the initial prognosis such that the oncologist has more comprehensive data on which to act. The ML model may remind the oncologist that certain follow up tests need to be performed at certain intervals during the course of the treatment to validate the efficacy of the course of treatment or to check whether mutations in the cancer genotype may have reduced the efficacy of the selected course of treatment indicating that different treatment options may be advisable. The ML model may also recommend new patient studies or pharmacological trials that are in progress the oncologist or referring physician may not be aware of that the patient may be eligible for based on the pathology data extracted from the medical reports associated with the patient's chart.

Further, the CRM system enables the use of automated models to allocate scarce marketing resources (e.g. callers) to markets (or other criteria), based on TAM (demand) and capacity (supply).

Further, the CRM system enables a platform standardizing de-identified patient data across RIS systems (e.g., Open Healthcare APIs) and constructing training datasets for continuous learning. A vendor agnostic integration engine is combined with radiology information systems to receive and parse appointment information and radiology reports in real-time. There are a number of different ways the ACT product receives and ingests data from different sources (RIS, EHR, EMR, MIS, etc.).

For example, data integration strategy options are provided as follows. In some embodiments, the data integration strategy comprises a VPN and read-only-access to full schema of Production RIS database. Once access is granted, everything is managed by WR engineering. SQL queries may be reviewed for performance safety. In some embodiments, the data integration strategy comprises a VPN and limited read-only-access via stored procedures or views to required data-fields, on Production RIS database. This enables separate stored procs for full update vs incremental update. This scales well, but IT needs to be heavily involved in writing the views/stored-procs, and then for every incremental data request. e.g. extra columns, missing timestamps for certain data fields, etc. In some embodiments, the data integration strategy comprises a VPN and read-only-access to full schema of 24-hour-delayed Reporting RIS Database. This is similar to the first option, but certain operational errors may arise (e.g. ACT could send an outreach text-message to a patient who already booked a screening in the last 24-hours), and time-sensitive aspects (e.g. ACT 2.0) may not be implemented correctly. Performance review may become less of an issue. In some embodiments, the data integration strategy comprises the second or third option, supplemented by an HL7 feed uploaded to a cloud storage. No incremental stored procs are needed for this option, yet allows time-sensitive aspects nicely. This may require higher effort to get started with compared to the other options.

The product automatically parses the different data fields received and converts the data to a standardized format. If data is received from 2 different sources (e.g., RIS & MIS) then the product joins data from these sources using multiple unique identifiers to ensure accuracy. As more information is captured through patient interactions through the ACT outreach, this data is added to the existing information filling any gaps, and creating a more complete dataset over time.

Further, dynamic de-identification and re-identification of data silos to enable data off-shoring of personal health information (PHI). Further, the CRM system enables a tool that estimates the value of a clinical dataset and enables participation in a marketplace.

Further, the CRM system enables data ingestion to be performed, which may include database integration, automated reports, and application programming interface (API) integration.

### Example 6—Customer Relationship Management System

Using systems and methods of the present disclosure, an ACT customer relationship management (CRM) system is configured as follows. System and methods are configured to analyze a group or individual patient's medical history and identify all possible healthcare procedures that they may be eligible for. Once an eligibility is identified, conduct patient outreach to the patient, doctor or other parties to encourage the adoption of the procedure. Procedures may include a consultation, diagnostic test, imaging exam, treatment, or any other intervention. The Documentation of patient medical history may include structured data (e.g., relational database systems), unstructured data (e.g., documents, PDFs), medical images, etc.

As an example, the ACT CRM system may be used to perform a prospective MSOP. If a patient is scheduled for any non-mammogram-screening exam, conduct analysis of her medical history as recorded in the radiology center's RIS systems, and identify if she may be eligible for a screening mammogram (e.g., gender is female, age is between 40-75, and no record of a screening done in the past 12 months in the RIS system).

As another example, the ACT CRM system may be used to perform a retrospective MSOP. Conduct analysis of a radiology center's EHR system to identify patients that have visited in the past only for non-mammogram-screening exams but are eligible for a screening mammogram.

As another example, the ACT CRM system may be used to conduct analysis of a lung-cancer patient's chart in the EMR of a primary care doctor or oncology office, and identify if he/she is eligible for a liquid biopsy test.

As another example, the ACT CRM system may be used to conduct analysis of a group or individual patient's medical history to identify all possible healthcare procedures that they may be eligible for. Conduct targeted advertising or marketing campaigns to such patients using online resources like Facebook Ads/Google AdWords. For example, based on the analysis, one could identify a demographic that is typically afflicted with a chronic condition and target that demographic with advertising for treatment options. This may be embodied as a EHR plugin at a doctor's office, where the system may recommend specific drugs or treatments to the doctor for prescribing to the patient.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A computer-implemented method for identifying subjects for a clinical procedure, comprising:
(a) retrieving, from a computer database, a first set of subject records, wherein the first set of subject records corresponds to a first set of subjects that are candidates for the clinical procedure;
(b) processing the first set of subject records using a trained machine learning algorithm to identify a second set of subjects and a second set of subject records associated with the second set of subjects, wherein the second set of subjects is a ranked subset of the first set of subjects, and wherein the second set of subject records is a subset of the first set of subject records; and
(c) electronically outputting the second set of subject records.

2. The method of claim 1, wherein the clinical procedure is a target screening exam, a diagnostic test, a prognostic test, a therapeutic intervention, or a prophylactic intervention.

3. The method of claim 2, wherein the clinical procedure is a diagnostic test for a clinical disease, disorder, or condition.

4. The method of claim 3, wherein the clinical disease, disorder, or condition is cancer.

5. The method of claim 4, wherein the cancer is breast cancer.

6. The method of claim 3, wherein the diagnostic test comprises obtaining a medical image of a test subject, and analyzing the medical image sample to determine a diagnosis of the clinical disease, disorder, or condition.

7. The method of claim 3, wherein the diagnostic test comprises obtaining a biological sample from a test subject, and assaying the biological sample to determine a diagnosis of the clinical disease, disorder, or condition.

8. The method of claim 1, wherein the first set of subjects previously received the clinical procedure.

9. The method of claim 1, wherein the second set of subjects is prioritized over other subjects of the first set of subjects with respect to the clinical procedure.

10. The method of claim 1, wherein (b) comprises processing a set of features of the first set of subject records using the trained machine learning algorithm to determine a score for each of at least a subset of the first set of subject records, and generating the second set of subject records based at least in part on the scores for the at least the subset of the first set of subject records.

11. The method of claim 10, wherein the score for a given subject record is indicative of a likelihood of compliance of a given subject with receiving the clinical procedure.

12. The method of claim 10, wherein a given subject record is selected for inclusion in the second set of subject records when the given subject record (i) has a score that is greater than a first pre-determined threshold and/or (ii) has a score that is less than a second pre-determined threshold.

13. The method of claim 10, wherein the set of features is selected from the group consisting of demographic characteristics, clinical characteristics, clinical history, and history of past outreach.

14. The method of claim 13, wherein the demographic characteristics are selected from the group consisting of age, gender, race, ethnicity, occupation, income, and education level.

15. The method of claim 13, wherein the demographic characteristics, clinical characteristics, clinical history, and/or history of past outreach are obtained from electronic medical records of subjects.

16. The method of claim 1, wherein the trained machine learning algorithm comprises a supervised machine learning algorithm.

17. The method of claim 16, wherein the supervised machine learning algorithm comprises a deep learning algorithm, a support vector machine (SVM), a neural network, or a Random Forest.

18. The method of claim 1, further comprising recruiting subjects to receive the clinical procedure based at least in part on the second set of subject records.

19. The method of claim 18, wherein recruiting the subjects comprises assigning the subjects to receive the clinical procedure at a clinical site.

20. The method of claim 19, further comprising selecting the clinical site from among a plurality of clinical sites based at least in part on an availability or load of the clinical site.

21. The method of claim 18, wherein recruiting the subjects comprises assigning the subjects to receive one of a set of alternative clinical procedures and/or treatments.

22. The method of claim 18, wherein recruiting the subjects comprises determining an optimal frequency, cadence, communication channel, and/or content for performing outreach attempts to the subjects.

23. The method of claim 18, wherein recruiting the subjects comprises determining an optimal frequency, cadence, communication channel, and/or content for temporally shifting patient populations.

24. The method of claim 1, further comprising transmitting an alert, notification, phone call, or reminder to at least one subject of the second set of subjects to have the clinical procedure performed.

25. A computer system for identifying subjects for a clinical procedure, comprising:
a database that is configured to store a first set of subject records, wherein the first set of subject records corresponds to a first set of subjects that are candidates for the clinical procedure; and
one or more computer processors operatively coupled to the database, wherein the one or more computer processors are individually or collectively programmed to:
(i) process the first set of subject records using a trained machine learning algorithm to identify a second set of subjects and a second set of subject records associated with the second set of subjects, wherein the second set of subjects is a ranked subset of the first set of subjects, and wherein the second set of subject records is a subset of the first set of subject records; and
(ii) electronically output the second set of subject records.

26.-48. (canceled)

49. A non-transitory computer readable medium comprising machine-executable code that, upon execution by one or more computer processors, implements a method for identifying subjects for a clinical procedure, the method comprising:
(a) retrieving, from a computer database, a first set of subject records, wherein the first set of subject records corresponds to a first set of subjects that are candidates for the clinical procedure;
(b) processing the first set of subject records using a trained machine learning algorithm to identify a second set of subjects and a second set of subject records associated with the second set of subjects, wherein the second set of subjects is a ranked subset of the first set of subjects, and wherein the second set of subject records is a subset of the first set of subject records; and
(c) electronically outputting the second set of subject records

50.-72. (canceled)
